(19) United States
(12) Patent Application Publication
(10) Pub. No.: US 2022/0280661 A1
(43) Pub. Date: Sep. 8, 2022

(54) GLUCOCORTICOID RECEPTOR RADIOLIGANDS AND METHODS FOR THEIR PREPARATION AND USE

(71) Applicant: The Regents of the University of California, Oakland, CA (US)

(72) Inventors: Michael Evans, Oakland, CA (US); Yangjie Huang, Oakland, CA (US); Christopher Drake, Oakland, CA (US)

(73) Assignee: The Regents of the University of California, Oakland, CA (US)

(21) Appl. No.: 17/737,649

(22) Filed: May 5, 2022

Related U.S. Application Data
(63) Continuation of application No. PCT/US2020/059406, filed on Nov. 6, 2020.
(60) Provisional application No. 62/932,849, filed on Nov. 8, 2019.

(30) Foreign Application Priority Data
Nov. 6, 2020 (WO) PCTUS2020059406

(51) Int. Cl. A61K 51/04

(52) U.S. Cl. CPC A61K 51/0455

(57) ABSTRACT

Disclosed herein are compounds useful for detection of glucocorticoid receptor expression and activity via positron emission tomography and related techniques. Methods for preparation and use of the compounds are also described.

### CROSS-REFERENCES TO RELATED APPLICATIONS

The present application is a Continuation of International Patent Application No. PCT/US2020/059406, filed Nov. 6, 2020, which claims priority to U.S. Provisional Patent Application No. 62/932,849, filed on Nov. 8, 2019, each of which is incorporated herein by reference in their entirety.

### STATEMENT AS TO RIGHTS TO INVENTIONS MADE UNDER FEDERALLY SPONSORED RESEARCH AND DEVELOPMENT

This invention was made with government support under grant no. W81XWH-15-1-0552 awarded by The United States Army Medical Research and Materiel Command, and grant no. R01 MH115043 awarded by The National Institutes of Health. The government has certain rights in the invention.

### BACKGROUND OF THE INVENTION

The glucocorticoid receptor (GR) regulates an exceptionally diverse spectrum of physiological processes, and is expressed in nearly every normal cell type. See, e.g., Kadmiel, et al. Trends in pharmacological sciences 2013, 34 (9), 518-30; Pujols, et al. American journal of physiology. Cell physiology 2002, 283 (4), C1324-31. As a nuclear hormone receptor (NHR), GR regulates biology primarily through an inducible transcriptional mechanism, in which cytosolic corticosteroid agonists bind the isoform GRα to trigger a conformational change that encourages nuclear translocation, homodimerization and DNA binding to regulate transcription. Agonist bound GRα can also regulate transcription through alternate mechanisms (e.g., tethering other transcription factors to DNA) or through non-genomic signaling. Moreover, other less abundant isoforms of GR, most notably the splice variant GRβ, may regulate additional dimensions of biology in a corticosteroid independent fashion. Further adding to the complexity, GR engages in both cell intrinsic and multiorgan endocrine signaling through secreted hormones (e.g., the hypothalamus-pituitary-adrenal axis).

Because of its seminal importance to the homeostasis of diverse cell types, dysregulation of GR signaling is accompanied by significant, and often highly debilitating, phenotypic changes. For instance, excess or deficient corticosteroid production and the concomitant dysregulation of cellular GRα activity lead to Cushing syndrome or Addison's disease, respectively. Moreover, tissue changes in GR expression—either losses or gains—have been implicated in the pathobiology of a wide spectrum of maladies, including mood disorders, glomerular diseases, and more recently, cancers (see, Kach, et al. Science translational medicine 2015, 7 (305), 305ps19).

Cell and genetically engineered animal models have been the mainstays for carefully articulating the abovementioned features of GR (patho)biology. Fully understanding GR's role in human physiology and disease, as well as developing next generation therapeutics to modulate GR, requires new technologies that can be applied to safely probe GR signaling in living subjects. Indeed, the lack of non-invasive biomarkers to interrogate GR signaling in the most clinically relevant settings has left the field to perform very suboptimal assays, for instance, analysis of human tissue for GR and target gene expression levels from samples collected at autopsy. See, Pandey, et al. Psychoneuroendocrinology 2013, 38 (11), 2628-39; Webster, et al. Molecular psychiatry 2002, 7 (9), 985-94, 924.

### BRIEF SUMMARY OF THE INVENTION

Provided herein are compounds according to Formula I, an pharmaceutically acceptable salts thereof, which can be used a glucocorticoid receptor (GR) ligands for in vivo study of GR expression and activity by techniques such as positron emission tomography (PET).

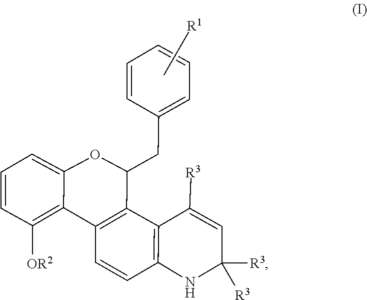

In compounds of Formula I:
R¹ is selected from the group consisting of halogen, C₁₋₆ alkyl, and C₆₋₁₂ aryl, each of which is optionally radiolabeled;
R² is C₁₋₆ alkyl, which is optionally radiolabeled; and
each R³ is independently C₁₋₆ alkyl.

Also provided herein are methods for preparing a compound according to Formula II:

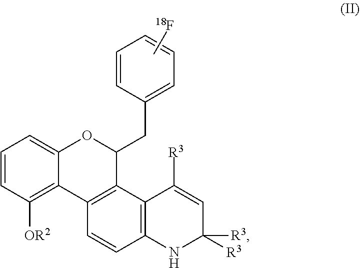

and pharmaceutically acceptable salt thereof. The methods include:
(a) forming a reaction mixture comprising an ¹⁸F-fluoride salt, a copper salt, and compound according to Formula IV:

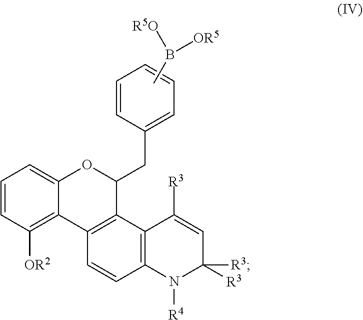

(b) maintaining the reaction mixture under conditions sufficient to form a compound according to Formula III:

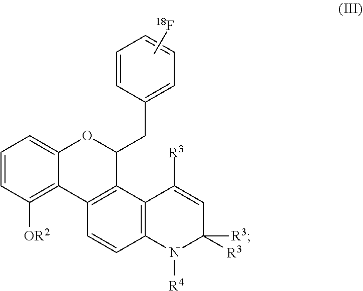

and
(c) converting the compound of Formula III to the compound of Formula II;
wherein:
R² is C₁₋₆ alkyl;
each R³ is independently C₁₋₆ alkyl;
R⁴ is an amine protecting group; and
each R⁵ is independently selected from the group consisting of hydrogen and C₁₋₆ alkyl, or
both R⁵ and the grouping —O—B—O— are taken together to form an optionally-substituted 5- or 6-membered ring.

In some embodiments, R¹ is —¹⁸F in radiolabeled compounds according to Formula I. Examples of radiolabeled GR ligands according to Formula I include, but are not limited to, 5-(4-(fluoro-¹⁸F)benzyl)-10-methoxy-2,2,4-trimethyl-2,5-dihydro-1H-chromeno[3,4-f]quinoline:

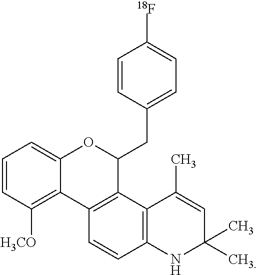 [SMILES: COc1cccc2c1-c1ccc3c(c1C(Cc1ccc([18F])cc1)O2)C(C)=CC(C)(C)N3]

Also provided herein are methods for detecting the expression of glucocorticoid receptor in a subject. The methods include: (i) administering to the subject an effective amount of a compound according to Formula I, or a pharmaceutically acceptable salt thereof, wherein at least one of R¹ and R² in the compound is radiolabeled, and wherein the compound is administered to the subject under conditions sufficient for binding of the compound to GR in the subject, and (ii) detecting the compound in the subject by positron emission tomography, thereby detecting the expression of GR in the subject.

GR expression may be detected, for example, in brain tissue, adipose tissue, kidney tissue, prostate tissue, or a combination thereof using the methods provided herein. In some embodiments, GR expression is detected in cancerous tissue in the subject. In some embodiments, the methods further include administering a therapeutically effective amount of a GR modulator to the subject, thereby treating cancer in the subject.

### BRIEF DESCRIPTION OF THE DRAWINGS

FIG. 1 shows the structure and numbering of the benzopyrano[3,4-f]quinoline and the structure of 5-(4-fluorobenzyl)-10-methoxy-2,2,4-trimethyl-2,5-dihydro-1H-chromeno[3,4-f]quinoline (YJH08).

FIG. 2A shows stacked semi-preparative UV HPLC traces exhibiting the retention of arylboronic acid pinacol ester starting material (4) and the Boc-protected aryl fluoride product (5). Below these analytical standards are shown the UV trace of the complex reaction mixture at 90 min and the corresponding trace collected using a radiation detector. A single product peak is observed on RAD-HPLC corresponding to the desired product, [18F]-5, while a minor UV peak is observed at the same retention time, as desired. The peak was collected and transferred immediately to a reaction vial for Boc deprotection.

FIG. 2B shows stacked semi-preparative UV HPLC traces exhibiting the retention of the Boc protected aryl fluoride (5) and the deprotected final product (6). Below the analytical standards are shown the UV trace of the complex reaction mixture at 30 min and the corresponding trace collected using a radiation detector. A single product peak is observed on RAD-HPLC corresponding to the desired product, [18F]-YJH08, while a minor UV peak is observed at the same retention time, as expected. After purification, the purity and identity of [18F]-YJH08 was confirmed using analytical HPLC with an isocratic gradient.

FIG. 3A shows a time activity curve from a dynamic PET scan shows the magnitude of radiotracer uptake and clearance in major tissue compartments of a male C57B16/J mouse (top panel). Coronal PET/CT images indicating the placement of the regions of interest to calculate the tissue uptake on the dynamic scan are show shown (bottom panels).

FIG. 3B shows representative sagittal and coronal slices of the mouse used to derive the time activity curves provide a visual rendering of relative radiotracer biodistribution in tissues.

FIG. 3C shows the results of a region of interest analysis on the supraspinal brown adipose tissue depot was used to calculate the depicted time activity curve.

FIG. 3D shows the results of a region of interest analysis performed on the PET data from the dynamic acquisition shows high and time dependent radiotracer accumulation in the brain.

FIG. 3E shows digital autoradiography and H&E staining of a frozen transverse section of the mouse brain show the relative distribution of [18F]-YJH08. Taken together, FIGS. 3A-3E demonstrate that [18F]-YJH08 targets GR rich tissues important to normal and disease physiology, including the brain.

FIG. 4A shows a dosing scheme applied with mice receiving vehicle or dexamethasone prior to the injection of [18F]-YJH08 into mice, represented as a timeline. Maximum intensity projections from two representative mice from the vehicle and dexamethasone cohorts are shown below the timeline.

FIG. 4B shows representative PET/CT slices showing the suppression of radiotracer uptake due to prior dexamethasone treatment in the brain (top white arrow), supraspinal brown fat (middle white arrow) and the kidneys (bottom white arrow). The bottom arrow in the top right panel indicates a submandibular region showing evidence of specific binding; this is tentatively interpreted to be anterior cervical or supraclavicular brown fat.

FIG. 4C shows a summary of the percent change in radiotracer uptake for mouse tissues in two separate cohorts comparing (1) the impact of dexamethasone treatment and (2) the impact of adrenalectomy (adx.) on [18F]-YJH08 radiotracer biodistribution. Taken together, FIGS. 4A-4C demonstrate that [18F]-YJH08 specifically binds to GR in vivo, and can be applied to measure receptor occupancy by endogenous or exogenous ligands.

FIG. 5A shows biodistribution data showing high [18F]-YJH08 uptake in PC3 tumors implanted in the subrenal capsule. Radiotracer uptake exceeded the extent observed in GR rich tissues like brain and lung. Moreover, that tumor uptake exceeded the level observed in bone—the most common site of prostate cancer metastasis—suggests this radiotracer could be implemented clinically. *P<0.01

FIG. 5B shows a merged image of the digital autoradiography and H&E of the affected kidney shows that there is relatively higher uptake in the tumor compared to normal kidney. The autoradiography is represented on a red (max) to blue (min) scale.

FIG. 5C shows a higher magnification image of the autoradiography, H&E and the fused images centered around the location of the PC3 tumor.

FIG. 6 shows a time activity curve from a dynamic PET scan in a wild type male C57B16/J mouse, demonstrating that (±)-¹¹C-YJH08 rapidly cleared from the serum, as expected, and the primary mode of clearance appeared to be hepatobiliary. The data also show saturable radiotracer accumulation in the brain and brown fat, two GR rich tissues. At right are shown representative sagittal and coronal slices of the mouse injected with (±)-¹¹C-YJH08 PET/CT.

FIG. 7A shows biodistribution data collected 20 min post injection, demonstrating the suppression of (±)-¹¹C-YJH08 uptake in several mouse tissues by dexamethasone (Dex, n=5/arm). Dex was administered via two routes involving oral gavage of 50 mg/kg for three days prior to radiotracer injection or administration of a water-soluble dexamethasone-cyclodextrin (CD) complex via intraperitoneal injection at 10 mg/kg one hour prior to radiotracer injection. *P<0.05. At right is shown the blocking data observed in the brain. *P<0.01

FIG. 7B shows the percent changes in radiotracer uptake per tissue among mice exposed to dexamethasone or Dex-CD versus vehicle.

FIG. 7C shows representative CT and PET/CT images demonstrating the suppression of (±)-¹¹C-YJH08 binding in selected organs in vivo by Dex treatment. Taken together, FIGS. 7A-7C demonstrate that (±)-¹¹C-YJH08 specifically binds GR in vivo

FIG. 8A shows an HPLC trace over a chiral stationary phase, providing the separation of racemic YJH08 into R and S isomers. The starred peaked at 29.1 min was isolated in and recrystallized in hexanes to give a single crystal.

FIG. 8B shows ³H-dexamethasone displacement curves on DU145 cells, demonstrating the relative affinities of (R)-YJH08, (S)-YJH08 and dexamethasone. Each ligand has an affinity <5 nM, and are equipotent. R² values for curve fitting were 0.99 (R), 0.94 (S), and 0.97 (dexamethasone).

FIG. 8C shows mouse biodistribution data, exhibiting the tissue uptake of R- and S-¹¹C-YJH08. The data were acquired 20 min post injection in male C57B16/J mice. Taken together, FIGS. 8A-8C show that YJH08 enantiomers have similar affinity for GR in vitro and similar biodistribution patterns in vivo.

FIG. 9A shows biodistribution data for uptake of ¹¹C-YJH02 in various mouse tissues. The data show tracer distribution in mice that received vehicle or dexamethasone-cyclodextrin at 10 mg/kg via intraperitoneal injection 1 hour prior to radiotracer injection. Radiotracer uptake was evaluated 20 min post injection. *P<0.01

FIG. 9B shows a comparison of ¹¹C-YJH02 and (±)-¹¹C-YJH08 uptake among tissues in normal mice. The uptake of ¹¹C-YJH02 was generally lower than (±)-¹¹C-YJH08. *P<0.05. Taken together, FIGS. 9A and 9B show the importance of the 4-fluoro-benzyl motif to specific GR binding in vivo.

FIG. 10A shows the structure of JG231, a recently developed potent HSP70 inhibitor with excellent pharmacokinetics and anti-tumor activity.

FIG. 10B shows immunoblot data showing that JG231 degrades GR in MCF7 and MDA MB 231, two human breast cancer cell lines. Cells were treated with the indicated dose for 24 hours.

FIG. 10C shows maximum intensity projections of viable mice showing the suppression of radiotracer uptake by JG231 treatment in the supraspinal brown fat depot and the omentum, but not in other tissues with high radiotracer uptake. At right is shown a maximum intensity projection of supraspinal BAT and muscle from a representative mouse in either treatment arm. Mice (n=5/treatment arm) were treated with JG231 or vehicle once daily for 3 days via intraperitoneal injection.

FIG. 10D shows biodistribution data showing the tissue uptake of ¹⁸F-YJH08 in mice from the treatment study. As predicted by the PET/CT data, JG231 suppressed radiotracer uptake exclusively in the supraspinal brown fat pad. *P<0.01. The biodistribution data are representative of two independent experiments.

FIG. 10E shows immunoblot data showing GR degradation in brown fat after treatment with JG231. GR levels were not impacted in liver, kidneys, or muscle, as predicted based on the radiotracer biodistribution data. Taken together, FIGS. 10A-TOE demonstrate the utility of ¹⁸F-YJH08 in revealing unexpected tissue specific GR degradation for the HSP70 inhibitor JG231.

FIG. 11 shows the structure of Formula I as described in more detail below.

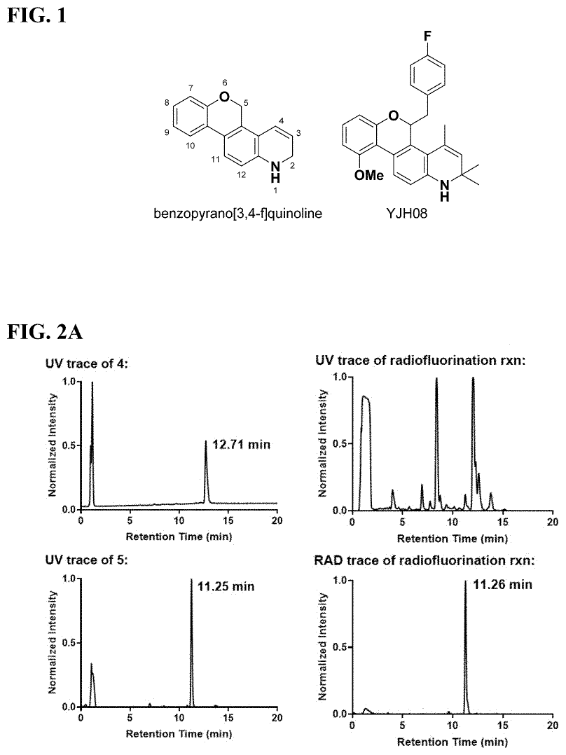

### DETAILED DESCRIPTION OF THE INVENTION

Described herein is the design, synthesis, and pharmacological evaluation of small molecule radiotracers including [¹⁸F]-5-(4-fluorobenzyl)-10-methoxy-2,2,4-trimethyl-2,5-dihydro-1H-chromeno[3,4-f]quinoline (termed “[18F]-YJH08”) that overcome the aforementioned challenges to measure GR expression levels in vivo with PET. [18F]-YJH08 has sub nM affinity for GR with at least 100 fold selectivity for GR over other subfamily C group 3 nuclear hormone receptors. High-yielding radiosynthesis can be achieved using Cu(II) mediated fluorination of an arylboronic acid pinacol ester precursor. Remarkably, [18F]-YJH08 rapidly and specifically bound to GR in nearly every normal mouse tissue tested in vivo. The level of radiotracer uptake was elevated in several tissues of great interest to the GR community, including the brain, adipose tissue, adrenals, and kidneys. The relevance of the radiotracer to studying aberrant GR expression in disease was demonstrated using a model of prostate cancer, a malignancy for which GR biomarkers are urgently needed to identify patients most likely to respond to GR antagonists in clinical trials.

### I. DEFINITIONS

As used herein, the term “alkyl,” by itself or as part of another substituent, refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆, and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc. Alkyl groups can be substituted or unsubstituted. Unless otherwise specified, “substituted alkyl” groups may be substituted with one or more groups selected from halo, hydroxy, amino, alkylamino, amido, acyl, nitro, cyano, and alkoxy.

As used herein, the term “aryl,” by itself or as part of another substituent, refers to an aromatic ring system having any suitable number of carbon ring atoms and any suitable number of rings. Aryl groups can include any suitable number of carbon ring atoms, such as C₆, C₇, C₈, C₉, C₁₀, C₁₁, C₁₂, C₁₃, C₁₄, C₁₅ or C₁₆, as well as C₆₋₁₀, C₆₋₁₂, or C₆₋₁₄. Aryl groups can be monocyclic, fused to form bicyclic (e.g., benzocyclohexyl) or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be substituted or unsubstituted. Unless otherwise specified, “substituted aryl” groups can be substituted with one or more groups selected from halo, hydroxy, amino, alkylamino, amido, acyl, nitro, cyano, and alkoxy.

As used herein, the terms “halogen” and “halo,” by themselves or as part of another substituent, refer to fluorine, chlorine, bromine and iodine.

As used herein, the term “hydroxy” refers to the moiety —OH.

As used herein, the term “oxo” refers to an oxygen atom that is double-bonded to a compound (i.e., O═).

As used herein, the term “amino” refers to a moiety —NR₂, wherein each R group is H or alkyl. An amino moiety can be ionized to form the corresponding ammonium cation. “Alkylamino” refers to an amino moiety wherein at least one of the R groups is alkyl.

As used herein, the term “amido” refers to a moiety —NRC(O)R or —C(O)NR₂, wherein each R group is H or alkyl.

As used herein, the term “acyl” refers to the moiety —C(O)R, wherein each R group is alkyl.

As used herein, the term “nitro” refers to the moiety —NO₂.

As used herein, the term “cyano” refers to a carbon atom triple-bonded to a nitrogen atom (i.e., the moiety —C≡N).

As used herein, the term “carboxy” refers to the moiety —C(O)OH.

As used herein, the term “radiolabeled” refers to a compound, such as a GR ligand, or a functional group within a compound that contains at least one atom that emits radiation such as alpha particles, beta particles, nucleons, electrons, positrons, neutrinos, or gamma rays. A positron emitter, for example, releases a positron and a neutrino resulting in conversion of a proton to a neutron. Positron emission typically occurs from proton-rich radionuclides, e.g., ⁶⁸Ga, ¹⁸F, ¹¹C and the like. Other radiolabels include, but are not limited to, phosphorus-32 (³²P), scandium-47 (⁴⁷Sc), cobalt-55 (⁵⁵Co), copper-60 (⁶⁰Cu), copper-61 (⁶¹Cu), copper-62 (⁶²Cu), copper-64 (⁶⁴Cu), gallium-66 (⁶⁶Ga), copper-67 (⁶⁷Cu), gallium-67 (⁶⁷Ga), rubidium-82 (⁸²Rb), yttrium-86 (⁸⁶Y), yttrium-87 (⁸⁷Y), iodine-124 (¹²¹), iodine-125 (¹²⁵I), and iodine-131 (¹³¹I)

As used herein, the term “salt” refers to a compounds comprising at least one cation (e.g., an organic cation or an inorganic cation) and at least one anion (e.g., an organic anion or an inorganic anion). In some embodiments, the salts may be an acid or base salt of GR ligand (e.g., a compound according to Formula I) or an active agent such as mifepristone. Acid salts of basic compounds include, but are not limited to, mineral acid salts (e.g., salts formed using hydrochloric acid, hydrobromic acid, phosphoric acid, and the like), organic acid salts (e.g., salts formed using acetic acid, propionic acid, glutamic acid, citric acid, and the like) salts, and quaternary ammonium salts (e.g., salts formed using methyl iodide, ethyl iodide, and the like). Acidic compounds may be contacted with bases to provide base salts such as alkali and alkaline earth metal salts (e.g., sodium, lithium, potassium, calcium, and magnesium salts), as well as ammonium salts (e.g., ammonium, trimethyl-ammonium, diethylammonium, and tris-(hydroxymethyl)-methyl-ammonium salts).

In some embodiments, the neutral forms of the active agents may be regenerated by contacting the salt with a base or acid and isolating the parent compound in the conventional manner if desired. In some embodiments, the parent form of the compound may differ from various salt forms in certain physical properties, such as solubility in polar solvents, but otherwise the salt forms may be equivalent to the parent form of the compound.

As used herein, the term “boronic acid” refers to the moiety —B(OH)₂. The terms “boronate” and “boronic acid ester” refer to a moiety —B(OR)₂, wherein each R is independently optionally substituted alkyl or optionally substituted aryl. Alternatively the boron atom and the —OR groups may be taken together to form a cyclic ester such as a pinacol ester.

As used herein, the term “pharmaceutically acceptable excipient” refers to a substance that aids the administration of a compound such as an active agent or diagnostic agent to a subject. By “pharmaceutically acceptable,” it is meant that the excipient is compatible with the other ingredients of the formulation and is not deleterious to the recipient thereof. It is understood, for example, that pharmaceutically acceptable excipients and salts are non-toxic. Useful pharmaceutical excipients include, but are not limited to, solvents, diluents, pH modifiers, and solubilizers.

As used herein, the terms “glucocorticoid receptor” and “GR” refer to a family of intracellular receptors also referred to as the cortisol receptor, which specifically bind to cortisol and/or cortisol analogs. Examples of glucocorticoid receptors include, but are not limited to, human GR (hGR) isoforms α and β. hGR isoforms are described, for example, by Nicolaides et al. (Steroids. 2010, 75(1): 1-12). The function of GRβ is largely unknown and thought to be independent of corticosteroid interactions. In contrast, GRα directly interacts with corticosteroids, and corticosteroid binding activates GRα transcription to regulate cell biology. GRβ is also expressed a low levels in normal mammalian tissues, while GRα is abundant and ubiquitously expressed. As used herein, the term “GR modulator” refers to a substance that affect the level of GR expression and/or activity. GR agonists, which promote GR activity, include but are not limited to hydrocortisone, dexamethasone, prednisone, and the like. GR antagonists, which inhibit GR activity, include but are not limited to mifepristone and ketoconazole.

As used herein, “cancer” and like terms refer to any member of a class of diseases or disorders characterized by uncontrolled division of cells and the ability of these cells to invade other tissues, either by direct growth into adjacent tissue through invasion or by implantation into distant sites by metastasis. Metastasis is defined as the stage in which cancer cells are transported through the bloodstream or lymphatic system. Cancers include, but are not limited to, carcinomas, lymphomas, leukemias, sarcomas, mesotheliomas, gliomas, germinomas, and choriocarcinomas.

As used herein, the terms “treat,” “treatment,” and “treating” refer to any indicia of success in the treatment or amelioration of an injury, pathology, condition, or symptom (e.g., cognitive impairment), including any objective or subjective parameter such as abatement; remission; diminishing of symptoms or making the symptom, injury, pathology or condition more tolerable to the patient; reduction in the rate of symptom progression; decreasing the frequency or duration of the symptom or condition; or, in some situations, preventing the onset of the symptom. The treatment or amelioration of symptoms can be based on any objective or subjective parameter; including, e.g., the result of a physical examination.

As used herein the term “effective amount” refer to a dose of a compound such as GR modulator that produces the outcome (e.g., a therapeutic effect, binding of an intended target, or an imaging result) for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); Goodman & Gilman's The Pharmacological Basis of Therapeutics, 11^{th} Edition, 2006, Brunton, Ed., McGraw-Hill; and Remington: The Science and Practice of Pharmacy, 21^{st} Edition, 2005, Hendrickson, Ed., Lippincott, Williams & Wilkins).

As used herein, the term “subject” refers to animals such as mammals, including, but not limited to, primates (e.g., humans), cows, sheep, goats, horses, dogs, cats, rabbits, rats, mice and the like.

### II. GLUCOCORTICOID RECEPTOR LIGANDS

The complexity of glucocorticoid receptor (GR) signaling cannot be measured with direct tissue analysis in living subjects, which has stifled understanding of GR's role in normal physiology or disease, and the pharmacology of next generation GR modulators. Advantageously, the compounds described herein provide for quantitative assessment of GR expression levels in real time among multiple tissues simultaneously. The compounds and methods of the present disclosure are therefore useful for unraveling the daunting complexity of GR signaling in vivo.

Accordingly, provided herein are compounds according to Formula I:

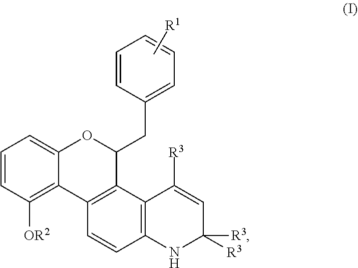

and pharmaceutically acceptable salts thereof, wherein:
R¹ is selected from the group consisting of halogen, C₁₋₆ alkyl, and C₆₋₁₂ aryl, each of which is optionally radiolabeled;
R² is C₁₋₆ alkyl, which is optionally radiolabeled; and
each R³ is independently C₁₋₆ alkyl.

In some embodiments, at least one of R¹ and R² is radiolabeled. In some embodiments, R¹ is labeled and R² is unlabeled. R¹ may be, for example, a radiolabeled halogen (e.g., radiolabeled fluorine such as fluorine-18 or radiolabeled iodine such as iodine-124). In some embodiments, R¹ is —¹⁸F. In some embodiments R¹ is radiolabeled halogen and R² is C₁₋₆ alkyl (e.g., methyl, ethyl, n-propyl, isopropyl, n-butyl, isobutyl, sec-butyl, tert-butyl, n-pentyl, branched pentyl, n-hexyl, or branched hexyl). In some embodiments, R² is methyl. R³ may be, for example, methyl in any of the foregoing embodiments.

In some embodiments, R² is labeled and R¹ is unlabeled. R² may be for example, radiolabeled C₁₋₆ alkyl (e.g., ¹¹C-containing methyl, ethyl, n-propyl, isopropyl, n-butyl, isobutyl, sec-butyl, tert-butyl, n-pentyl, branched pentyl, n-hexyl, or branched hexyl). In some embodiments, R² is —¹¹CH₃. In some embodiments, R² is radiolabeled alkyl (e.g., —¹¹CH₃), and R¹ is unlabeled halogen. In some embodiments, R² is radiolabeled alkyl (e.g., —¹¹CH₃), and R¹ is C₁₋₆ alkyl (e.g., methyl, ethyl, n-propyl, isopropyl, n-butyl, isobutyl, sec-butyl, tert-butyl, n-pentyl, branched pentyl, n-hexyl, or branched hexyl). In some embodiments, R² is radiolabeled alkyl (e.g., —¹¹CH₃), and R¹ is unlabeled C₆₋₁₂ aryl (e.g., phenyl, biphenyl, or naphthyl). R³ may be, for example, methyl in any of the foregoing embodiments.

In some embodiments, the compound has a structure according to Formula Ia:

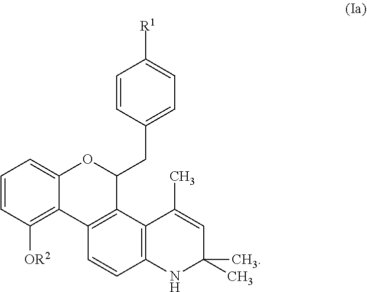

In some embodiments, R¹ is —¹⁸F and R² is —CH₃ in compounds according to Formula I and Formula Ia. In some embodiments, R¹ is —F and R² is —¹¹CH₃ in compounds according to Formula I and Formula Ia. In some embodiments, R¹ is —Cl, —Br, —I or phenyl in compounds according to Formula I and Formula Ia, and R² is —¹¹CH₃.

In some embodiments, the compound is selected from:

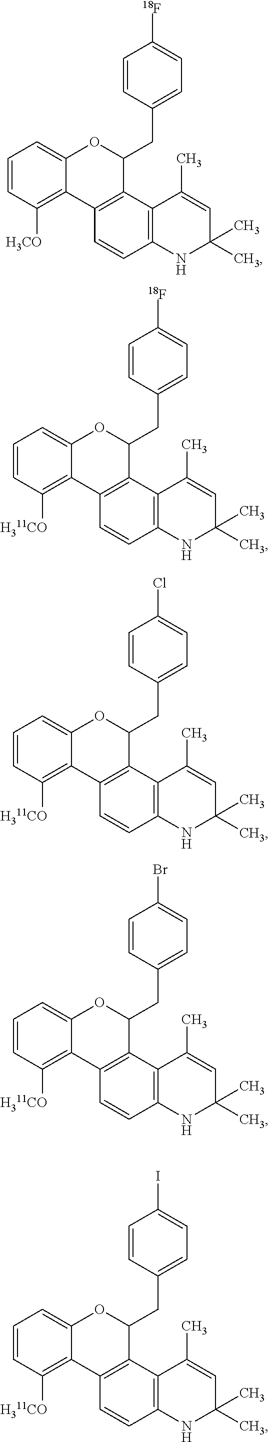

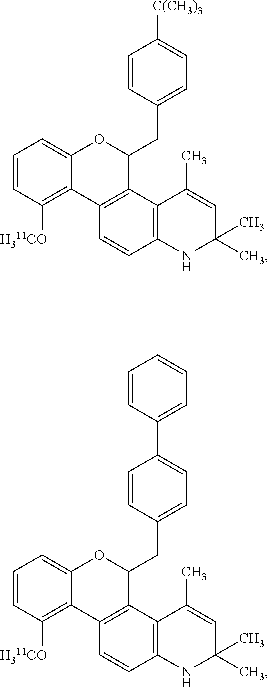

and pharmaceutically acceptable salts thereof.

Compounds according to the present disclosure may be synthesized from starting materials that are commercially available or those that can be prepared according to known methods, including those described in Fiesers' Reagents for Organic Synthesis Volumes 1-28 (John Wiley & Sons, 2016), by March (Advanced Organic Chemistry 6^{th} Ed. John Wiley & Sons, 2007), and by Larock (Comprehensive Organic Transformations 3^{rd} Ed. John Wiley & Sons, 2018). For example, compounds may be synthesized as shown in Scheme 1. (2,6-Dimethoxyphenyl)boronic acid and methyl 2-bromo-5-nitrobenzoate may be reacted in a Suzuki type palladium-catalyzed coupling reaction to provide biphenyl (i), which can be converted to benzocoumarin (ii) upon cleavage of the methyl ether groups (e.g., with boron tribromide). Reaction with alkylating reagent R²—X (wherein X is, e.g., halogen or sulfonate) can be employed to obtain intermediate (iii) prior to reduction of the nitro group to obtain aniline (iv). The aniline can then be reacted, for example, with enone (v) to provide dihydroquinolinone (vi). Alternatively, in cases where each R³ is methyl, the aniline may be reacted with acetone to provide dihydroquinolinone (vi). Reduction and alkylation of the quinolinone provides dihydroquinolinol ether (vii) where R⁷ is, for example, C₁₋₆ alkyl.

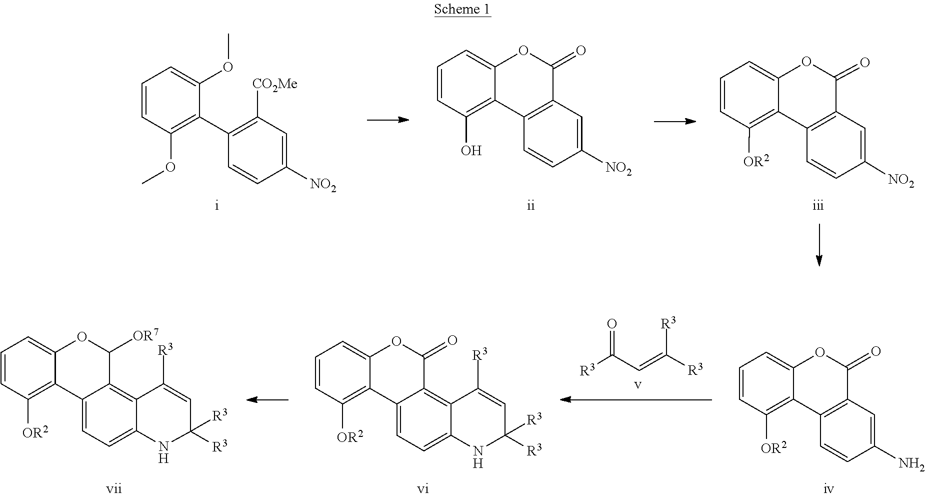

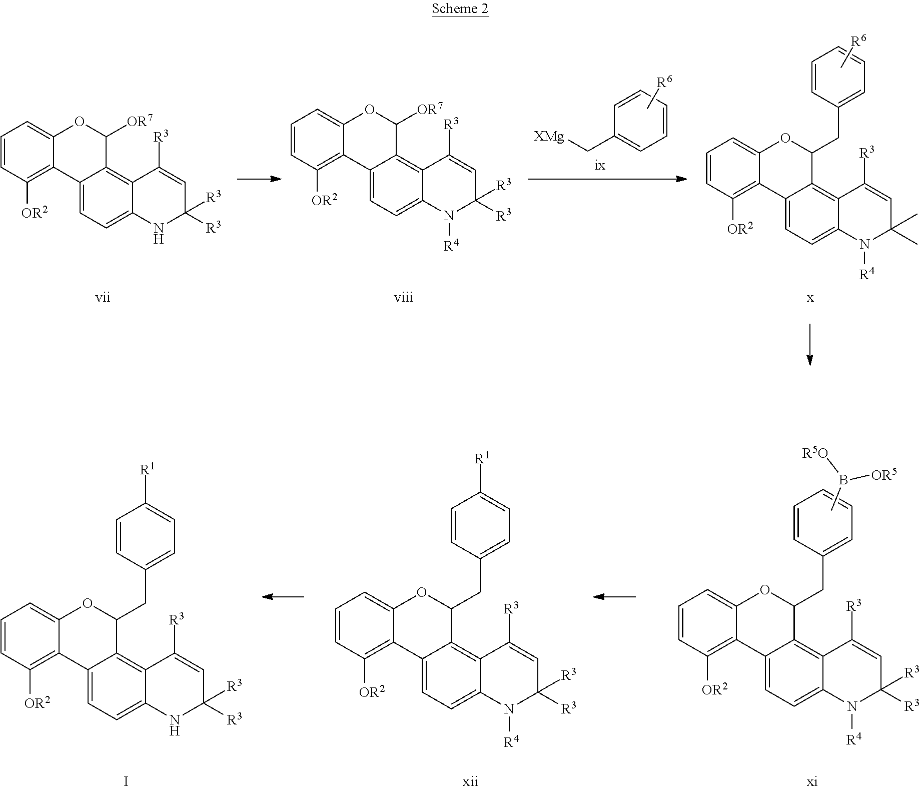

As shown in Scheme 2, dihydroquinolinol ether (vii) may be protected, e.g., such than R⁴ in protected intermediate (viii) is Boc, Fmoc, or another amine-protecting group. The protected intermediate can be reacted with Grignard reagent (ix), wherein R⁶ is, e.g., halogen or sulfonate, and then with diboron reagent (R⁵O)₂B—B(OR⁵)₂ to provide boronate (xi). R³ may be hydrogen and C₁₋₆ alkyl, or —O—B—O— may form a cyclic boronate ester such as a pinacol boronate ester. Boronate (xi) can be converted to a radiolabeled ligand in protected form (e.g., wherein R¹ is ¹⁸F) prior to deprotection to afford the radiolabeled compound according to Formula I.

Accordingly, also provided herein are methods for preparing a compound according to Formula II:

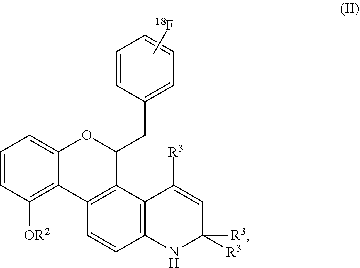

and pharmaceutically acceptable salts thereof. The methods include:
(a) forming a reaction mixture comprising an ¹⁸F-fluoride salt, a copper salt, and compound according to Formula IV:

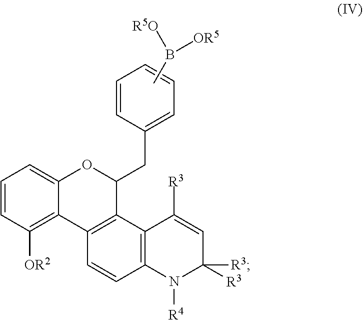

(b) maintaining the reaction mixture under conditions sufficient to form a compound according to Formula III:

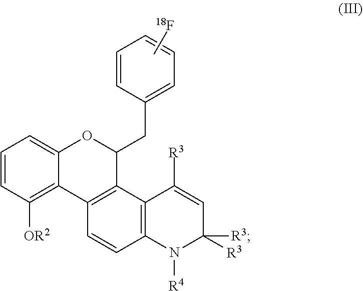

and
(c) converting the compound of Formula III to the compound of Formula II;
wherein:
R² is C₁₋₆ alkyl;
each R³ is independently C₁₋₆ alkyl;
R⁴ is an amine protecting group; and
each R⁵ is independently selected from the group consisting of hydrogen and C₁₋₆ alkyl, or
both R⁵ and the grouping —O—B—O— are taken together to form an optionally-substituted 5- or 6-membered ring.

Synthetic methods used to generate reactive [¹⁸F]fluoride and salts thereof are well known in the art and can be found in, for example, Guillaume et. al, Appl. Radiat. Isot. 1991, 42, 749-762; Ding, Y.-S., et al. J. Fluorine Chem. 1990, 48, 189-206; Ding Y.-S., et al. J Labelled Cmpd. Radiopharm. 1997, 39, 303-318; Synthesis and Evaluation of 6-[18F]Fluoro-3-(2(S)-azetidinylmethoxy)pyridine as a PET Tracer for Nicotinic Acetylcholine Receptors Nuclear Medicine & Biology, Vol. 27, pp. 381-389, 2000; Lee, E. et al. J. Am. Chem. Soc., 2012, 134, 17456-17458. Typically, the [¹⁸F]fluoride is prepared from ¹⁸O-enriched water by the ¹⁸O(p,n)¹⁸F-nuclear reaction using a cyclotron and a silver-bodied target. [¹⁸F]fluoride is generally isolated from the (p,n)-nuclear reaction as a salt, such as Na¹⁸F, K¹⁸F, Cs¹⁸F, tetraalkylammonium [¹⁸F]fluoride, or tetraalkylphosphonium [¹⁸F]fluoride, and further steps are conducted using a suitable solvent under conditions sufficient to form the desired labeled products, as described in further detail below.

To increase the reactivity of the [¹⁸F]fluoride, a phase transfer catalyst such as an aminopolyether or crown ether (e.g., 4,7,13,16,21,24 hexaoxa-1,10-diazabicyclo[8,8,8]hexacosane, Kryptofix 2.2.2) may be added. The addition of Kryptofix or a crown ether can enhance the nucleophilicity of the fluoride anion by sequestering metal counter cations (e.g., sodium, potassium, and the like). In some cases, a base such as sodium carbonate or potassium carbonate can be used to suppress the generation of hydrogen fluoride, while supplying an optimal counter anion for the aminopolyether or crown ether selected. Optionally, a free radical trap may be used to improve fluoridation yields, as described in WO 2005/061415. As used herein the term “free radical trap” refers to any agent that interacts with free radicals and inactivates them. A suitable free radical trap for this purpose may be selected from 2,2,6,6-tetramethylpiperidine-N-oxide (TEMPO), 1,2-diphenylethylene (DPE), ascorbate, para-amino benzoic acid (PABA), α-tocopherol, hydroquinone, di-t-butyl phenol, β-carotene and gentisic acid.

In some embodiments, the nucleophilic displacement reaction is conducted in an aprotic solvent selected from dichloromethane, tetrahydrofuran, ethyl acetate, acetone, dimethylformamide, acetonitrile, dimethyl sulfoxide, propylene carbonate, and combinations thereof. In some embodiments, the aprotic solvent is dichloromethane, dimethyl sulfoxide, dimethylformamide, or tetrahydrofuran. In some embodiments, the nucleophilic displacement reaction is performed in dimethyl sulfoxide or dimethylformamide. In some embodiments, the nucleophilic displacement reaction is performed in dimethyl sulfoxide.

The reaction between a compound of Formula IV and [¹⁸F]fluoride may be performed for any length of time suitable for substitution of the boronate moiety, typically ranging from several seconds to several minutes. For example, the reaction mixture is maintained for a period of time ranging from about 15 seconds to about 60 minutes. In some embodiments, the reaction mixture is maintained for about 15 seconds, for about 30 seconds, or for about 1, 2, 3, 4, 5, 10, 15, 20, 25, 30, 45, or 60 minutes. The reaction may be conducted at any suitable temperature, typically ranging from ambient temperatures to reflux temperatures of a particular solvent. For example, the reaction mixture is maintained at a temperature ranging from about 15° C. to about 200° C. In some embodiments, the reaction mixture is maintained at about 15° C., 20° C., 30° C., 40° C., 50° C., 60° C., 70° C., 80° C., 100° C., 120° C., 140° C., 160° C., 180° C., or about 200° C.

A non-radioactive intermediate according to Formula IV may be contacted with any amount of [¹⁸F]fluoride suitable for substitution of the boronate with [¹⁸F]fluoride. In some embodiments, about 0.1 to about 50 molar equivalents of the [¹⁸F]fluoride material with respect to the compound of Formula IV are employed.

Amine protecting group R⁴ can be removed using known synthetic procedures. As used herein, the term “amine protecting group” refers to a chemical moiety that renders an amino group unreactive, but is also removable so as to restore the amino group. Examples of amine protecting groups include, but are not limited to, benzyloxycarbonyl, 9-fluorenylmethyloxy-carbonyl (Fmoc), tert-butyloxycarbonyl (Boc), allyloxycarbonyl (Alloc), acetamido, phthalimido, and the like. Other amine protecting groups are known to those of skill in the art including, for example, those described by Green and Wuts (Protective Groups in Organic Synthesis, 4^{th} Ed. 2007, Wiley-Interscience, New York). If desired or necessary, purification of the final radiolabeled product can be conducted (e.g., via reverse-phase liquid chromatography).

### III. PHARMACEUTICAL COMPOSITIONS AND METHODS OF ADMINISTRATION

Also provided herein are compositions comprising one or more compounds as described above and one or more pharmaceutically acceptable excipients. Liquid form preparations include solutions, suspensions, and emulsions. Such formulations may be administered by injection, that is, intravenously, intramuscularly, intracutaneously, subcutaneously, intraduodenally, or intraperitoneally.

Formulations for administration will commonly comprise a solution of the GR ligand dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of GR modulator in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension.

Oil suspensions can be formulated by suspending a GR modulator in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. Such formulations can also contain a demulcent, a preservative, or a coloring agent.

One of skill in the art will appreciate that still other modes of administration (e.g., oral, topical, parenteral, nasal, or pulmonary administration) may be utilized, particularly in cases where non-radiolabeled compounds are to be administered in connection with the treatment of GR-mediated diseases or conditions (e.g., a cancer or a mood disorder).

### IV. METHODS FOR GLUCOCORTICOID RECEPTOR DETECTION

Also provided herein are methods for detecting the expression of glucocorticoid receptor (GR) in a subject. The methods include:
(i) administering to the subject an effective amount of a compounds according to Formula I as described above, or a pharmaceutically acceptable salt thereof,
wherein at least one of R¹ and R² in the compound is radiolabeled, and wherein the compound is administered to the subject under conditions sufficient for binding of the compound to GR in the subject; and
(ii) detecting the compound in the subject by positron emission tomography, thereby detecting the expression of GR in the subject.

In some embodiments, GR expression is detected in brain tissue, adipose tissue, kidney tissue, or a combination thereof in the subject. In some embodiments, GR expression is detected in brain tissue.

In some embodiments, GR expression is detected in cancerous tissue in the subject. Cancerous tissue may include, for example, prostate cancer, breast cancer, ovarian cancer, non-small cell lung cancer. In some embodiments, GR expression is detected in prostate cancer in the subject.

In some embodiments, the method further includes administering to the subject a therapeutically effective amount of a GR modulator, thereby treating cancer in the subject. Examples of GR modulators include, but are not limited to, dexamethasone, mifepristone, prednisolone, dagrocorat, fosdagrocorat, mapracorat, dissociated glucocorticoids (e.g., RU24858, RU24782, and the like), 21-hydroxy-6,19-oxidoprogesterone, avicin D, and benzothiazole-dhodacyanines (e.g., 3-((5-bromothiophen-2-yl)methyl)-2-((Z)-((E)-5-(6-chloro-3-methylbenzo[d]thiazol-2(3H)-ylidene)-3-ethyl-4-oxothiazolidin-2-ylidene)methyl)thiazol-3-ium chloride, also referred to as JG-231, and the like).

In some embodiments, the compound is:

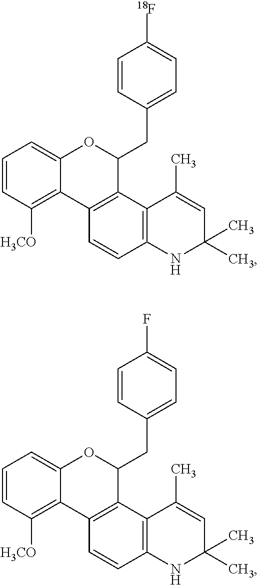 [SMILES: COc1cccc2c1-c1ccc3c(c1C(Cc1ccc([18F])cc1)O2)C(C)=CC(C)(C)N3 ; COc1cccc2c1-c1ccc3c(c1C(Cc1ccc(F)cc1)O2)C(C)=CC(C)(C)N3]

or a pharmaceutically acceptable salt thereof.

Compounds of the invention are generally administered in amounts sufficient to provide PET images of good quality (e.g., images that show the presence of GR in the brain of the subject or at another location in the subject's body). In general, the compound is administered at a dose ranging from about 0.1 milligrams to about 1000 milligrams per kilogram of a subject's body weight (i.e., about 0.1-1000 mg/kg). In some embodiments, the compound is administered at a dose ranging from about 0.1 milligram to about 200 milligrams per kilogram of a subject's body weight (i.e., about 1-100 mg/kg). The dose can be, for example, about 0.1-1000 mg/kg, or about 1-10 mg/kg, or about 10-50 mg/kg, or about 25-50 mg/kg, or about 50-75 mg/kg, or about 75 mg/kg, or about 1-100 mg/kg, or about 1-500 mg/kg, or about 25-250 mg/kg, or about 50-100 mg/kg. The dose can be about 1, 2, 3, 4, 5, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950 or 1000 mg/kg. In some embodiments, the compound in step (i) is administered to the subject in an amount ranging from about 0.1 mg/kg to about 200 mg/kg.

Typically, the compounds will be administered in amounts to provide radiation doses range from about 0.1 megabecquerel per kilogram (MBq/kg) to about 10 MBq/kg based on the body weight of the subject. For example, a compound can be administered in an amount sufficient to provide a radiation dose ranging from about 0.75 MBq/kg to about 3 MBq/Kg. Detection of positron emission and imaging can be conducted using instrumentation and techniques that are known in the art and described, for example, in U.S. Pat. Nos. 10,036,817; 9,872,664; 8,993,971; 8,084,742; 7,504,634; and 6,072,177, which patents are incorporated herein by reference. While the compounds will generally be detected via PET following administration to the subject, the methods can also be used in combination with any other methods of diagnostic imaging including, but not limited to, x-ray computed tomography (CT), magnetic resonance imaging (MRI), and functional magnetic resonance imaging (fMRI), ultrasound, and single photon emission computed tomography (SPECT).

### V. EXAMPLES

### Example 1. Compound Synthesis

All reactions were performed under an argon atmosphere using anhydrous solvents obtained from commercial suppliers in oven-dried round-bottom flasks containing Teflon coated stirrer bars, unless otherwise noted. All anhydrous solvents used were purchased from Sigma-Aldrich and used without further purification. Solvents to be employed in flash column chromatography and reaction work-up procedures were purchased from either Sigma-Aldrich or Fisher Scientific. All other reagents were obtained commercially and used without further purification, unless otherwise stated. Air and/or moisture sensitive reagents were transferred via syringe and were introduced into reaction vessels through rubber septa. Reactions were monitored using thin layer chromatography (TLC), performed on 0.25-mm EMD pre-coated glass-backed silica gel 60 F-254 plates. Column chromatography was performed on Silicycle Sili-prep cartridges using a Biotage Isolera Four automated flash chromatography system. Compounds were visualized under UV light or through staining with permanganate, or most preferably for trioxolane analogs, Seebach's “Magic” stain (composed of the following: 2.5 g phosphomolybdic acid, 1.0 g cerium sulfate, 6 mL concentrated sulfuric acid, and 94 mL water). Solution containing crude reaction mixtures, as well as those solutions obtained upon work-up of the reaction, and chromatography fractions were first concentrated by rotary evaporation at temperatures under 40° C., at 20 Torr then subsequently placed under Hi-Vac at 0.5 Torr unless otherwise indicated. It is imperative to maintain water bath temperatures <40° C. during rotary evaporation due to the thermal instability of trioxolanes at higher temperatures.

NMR spectra were recorded on a Bruker Advance III 400 MHz spectrometer at the UCSF NMR lab, and spectra were analyzed using Mestrelab software. Data for ¹H NMR spectra are reported in terms of chemical shift (6, ppm), multiplicity, coupling constant (Hz), and integration. Data for ¹³C NMR spectra are reported in terms of chemical shift (6, ppm), with multiplicity and coupling constants in the case of C—F coupling. The following abbreviations are used to denote the multiplicities: s=singlet; d=doublet; dd=doublet of doublets; dt=doublet of triplets; dq=doublet of quartets; ddd=doublet of doublet of doublets; t=triplet; td=triplet of doublets; tt=triplet of triplets; q=quartet; qd=quartet of doublets; quin=quintet; sex=sextet; m=multiplet.

LC-MS and compound purity were determined using Waters Micromass ZQ 4000, equipped with a Waters 2795 Separation Module, Waters 2996 Photodiode Array Detector, and a Waters 2424 ELSD. Separations were carried out with an XBridge BEH C18, 3.5 μm, 4.6×20 mm column, at ambient temperature (unregulated) using a mobile phase of water-methanol containing a constant 0.1% formic acid. High resolution mass spectra were recorded at the QB3/Chemistry Mass Spectrometry Facility at UC Berkeley.

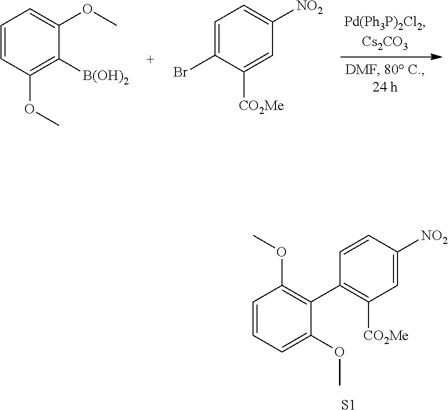

Methyl 2-(2,6-dimethoxyphenyl)-5-nitrobezoate (S1). A dry, two-necked 500 mL round bottom flask equipped with a mechanical stirrer was sequentially charged with 1,3-dimethoxyphenylboronic acid (10.9 g, 60 mmol), methyl 5-nitro-2-bromobenzate (12.9 g, 50 mmol), Pd(Ph₃P)₂Cl₂ (2.1 g, 3.0 mmol), Cs₂CO₃ (58.6 g, 180 mol) and dry DMF (200 mL). The reaction mixture was heated to 80° C. and vigorously stirred for 24 h, then cooled to r.t. and treated with water (300 mL) and EtOAc (300 mL). The layers were separated, and the aqueous layer was extracted with EtOAC (100 mL×3). The organics fractions were combined and washed with brine (100 mL×3), dried (Na₂SO₄) and concentrated under reduced pressure to removed most of solvent. As a yellow solid began to precipitate from solution, the evaporation was stopped, and the remaining mixture was transferred to a −20° C. freezer (−20° C.). After 2 h the yellow solid was collected by filtration and washed by hexanes to provide S1 (14.3 g, 45 mmol, 75%). ¹H NMR (400 MHz, DMSO) δ 8.52 (d, J=2.5 Hz, 1H), 8.37 (dd, J=8.5, 2.5 Hz, 1H), 7.58 (d, J=8.5 Hz, 1H), 7.36 (t, J=8.4 Hz, 1H), 6.75 (d, J=8.4 Hz, 2H), 3.65 (s, 6H), 3.65 (s, 3H).

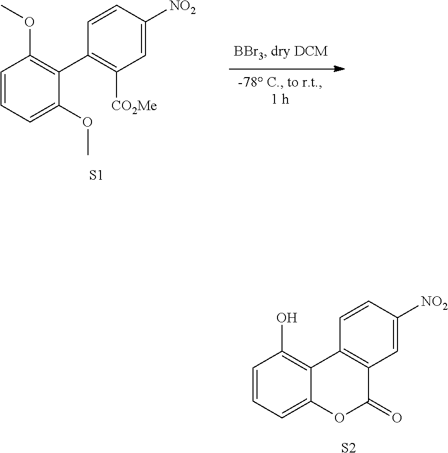

1-Hydroxy-8-nitro-6H-dibenzo[b,d]pyran-6-one (S2). To a solution of S1 (14.3 g, 45 mol) in dry CH₂Cl₂ at −78° C. was added BBr₃ dropwise (33.5 g, 135 mol). After the addition was complete, the reaction was slowly warmed to r.t. The solution turned dark red, and a solid began to precipitate from solution. This reaction mixture was further stirred at r.t. for 1 h and then re-cooled to −78° C. The reaction was quenched carefully with anhydrous MeOH (100 mL). Upon addition of MeOH, the red disappeared, and a bright yellow solid precipitated from solution. The yellow precipitate was collected by filtration to provide pure product S2 (10.4 g, 40.5 mmol, 90%). ¹H NMR (400 MHz, DMSO) δ 11.48 (br, s, 1H), 9.31 (d, J=9.2 Hz, 1H), 8.86 (d, J=2.6 Hz, 1H), 8.66 (dd, J=9.2, 2.7 Hz, 1H), 7.47 (t, J=8.2 Hz, 2H), 6.96 (dd, J=8.3, 1.0 Hz, 1H), 6.92 (dd, J=8.2, 1.1 Hz, 1H).

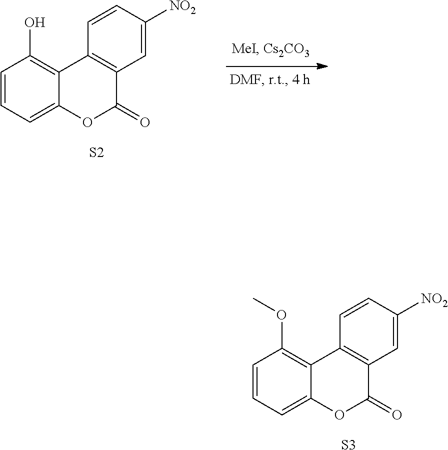

1-Methoxy-8-nitro-6H-dibenzo[b,d]pyran-6-one (S3). A dry, two-necked 1 L round bottom flask equipped with a mechanical stirrer was charged with S2 (25.7 g, 0.1 mol) and anhydrous cesium carbonate (65.1 g, 0.2 mol) in DMF (300 mL). Methyl iodide (56.7 g, 0.4 mol) was then added in a dropwise fashion via syringe. After 4 h, the reaction was quenched by addition of H₂O (200 mL). A solution of 50% EtOAc/hexanes (200 mL) was added, and the mixture was stirred 15 min. The mixture was filtered, washed with water (100 mL) and dried under vacuum to afford S3 (26.8 g, 99.0 mmol, 99%) as a yellow solid. ¹H NMR (400 MHz, DMSO) δ 9.22 (d, J=9.2 Hz, 1H), 8.90 (d, J=2.7 Hz, 1H), 8.66 (dd, J=9.2, 2.7 Hz, 1H), 7.66 (t, J=8.4 Hz, 1H), 7.18 (d, J=8.3 Hz, 1H), 7.12 (d, J=7.6 Hz, 1H), 4.10 (s, 3H).

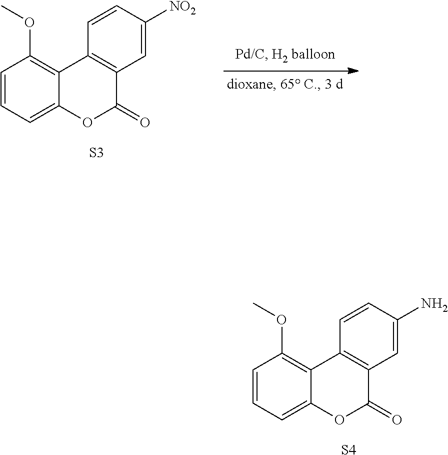

1-Methoxy-8-amino-6H-dibenzo[b,d]pyran-6-one (S4). To a suspension of S3 (26.8 g, 99.0 mmol) in dry dioxane (600 mL) at room temperature was added 10% palladium on carbon (1.17 g). The reaction was heated at 65° C., and a H₂(g) was introduced with a balloon. After 3 d, the reaction contents were filtered through Celite while still hot, and the Celite was washed with hot dioxane (3×60 mL). The filtrate was partially concentrated until the aniline S4 precipitated from solution (21.5 g, 89.1 mmol, 90%). ¹H NMR (400 MHz, DMSO) δ 8.70 (d, J=8.9 Hz, 1H), 7.45 (d, J=2.7 Hz, 1H), 7.34 (t, J=8.3 Hz, 1H), 7.11 (dd, J=8.9, 2.7 Hz, 1H), 7.02-6.95 (m, 2H), 5.88 (br, s, 2H), 4.00 (s, 3H).

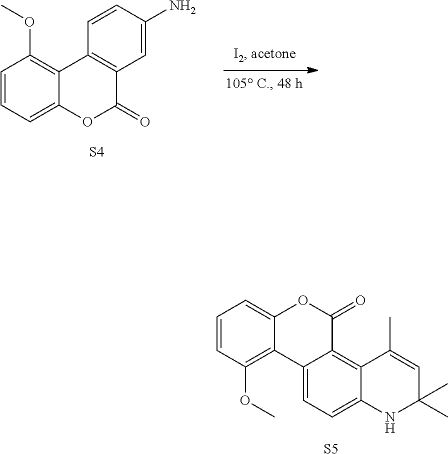

2,5-Dihydro-10-methoxy-5-oxo-2,2,4-trimethyl-1H-[1]benzopyrano [3,4-f]quinoline (S5). A solution of S4 (4.0 g, 16.6 mmol) and iodine (1.68 g, 6.64 mmol) in dry acetone (380 mL) placed into a 1 L high pressure vessel. The reaction mixture was stirred for 48 h at 105° C., cooled to room temperature and concentrated under vacuum. The resulting brown oil was purified by flash chromatography using silica gel eluting with 15 to 30% EtOAc in hexanes to give the desired compound S5 (2.29 g, 7.14 mmol, 43%). ¹H NMR (400 MHz, DMSO) δ 8.64 (d, J=9.0 Hz, 1H), 7.34 (t, J=8.3 Hz, 1H), 7.11 (d, J=9.0 Hz, 1H), 6.98 (d, J=8.4 Hz, 1H), 6.94 (dd, J=8.2, 0.9 Hz, 1H), 6.89 (br, s, 1H), 5.44 (br, s, 1H), 3.99 (s, 3H), 1.93 (d, J=1.0 Hz, 3H), 1.22 (s, 6H).

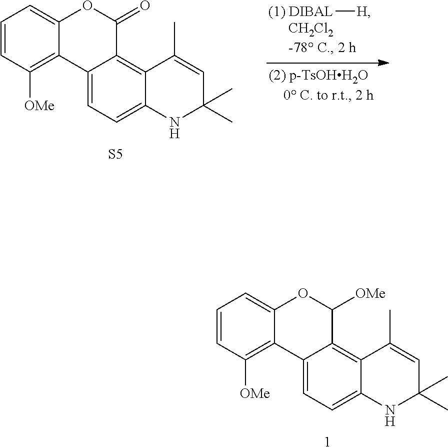

5,10-Dimethoxy-2,5-dihydro-2,2,4-trimethyl-1H-[1]benzopyrano[3,4-f]quinolone (1). A solution of the hemiacetal lactol S5 (11.5 g 35.8 mmol) in dry CH₂Cl₂ (200 mL) was slowly added 1M DIBAL-H in hexane (89.5 mL, 89.5 mmol) at −78° C. under Ar (g). The solution turned orange and was stirred at −78° C. for 2 h. The reaction was quenched with 300 mL EtOAc, and 200 mL of saturated aqueous Rochelle's salt was added. The mixture was stirred vigorously for overnight at room temperature. The aqueous layer was then separated and further washed with EtOAc. The combined organic layers were then washed with brine, dried with Na₂SO₄, and concentrated to give the lactol (9.6 g, 29.7 mmol, 83%) as a light yellow solid. The lactol (9.6 g, 29.7 mmol) was then dissolved in 200 mL of MeOH at 0° C., and a solution of p-TsOH.H₂O (1.87 g) in MeOH was slowly added. The reaction was stirred for 30 min at 0° C. The reaction was slowly brought to room temperature and stirred for 1 h. The reaction mixture was concentrated under reduced pressure to remove most of MeOH. Then EtOAc (200 mL) was added and the solution was washed with saturated aqueous NaHCO₃, and brine. The solution was dried with Na₂SO₄ and concentrated. The resulting brown oil was purified by flash chromatography using silica gel with 0 to 12% EtOAc in hexane to give the methyl acetal 1 (8.61 g, 25.5 mmol, 86%) as an off-white powder. ¹H NMR (400 MHz, DMSO) δ 7.99 (d, J=8.6 Hz, 1H), 7.11 (t, J=8.1 Hz, 1H), 6.76 (d, J=7.9 Hz, 1H), 6.71 (d, J=7.9 Hz, 1H), 6.67 (d, J=8.6 Hz, 1H), 6.20 (s, 2H), 5.45 (s, 1H), 3.87 (s, 3H), 3.33 (s, 3H), 2.17 (s, 3H), 1.27 (s, 3H), 1.07 (s, 3H).

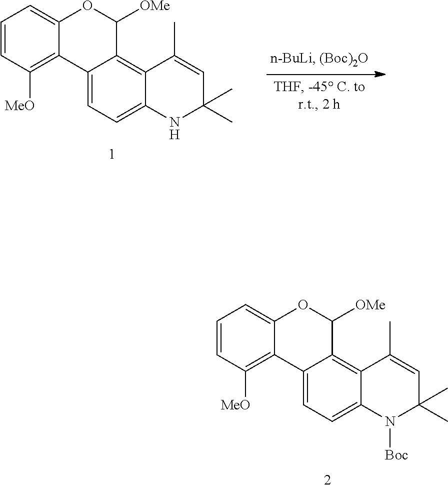

tert-Butyl-5,10-dimethoxy-2,2,4-trimethyl-2,5-dihydro-1H-chromeno[3,4-f]quinoline-1-carboxylate (2). A solution of methyl acetal 1 (1 g, 2.96 mmol) in anhydrous tetrahydrofuran (70 mL) was cooled to −45° C., whereupon n-butyllithium (1.25 mL of 2.5 M solution in hexanes, 3.11 mmol) was added dropwise over 30 min. The mixture was stirred at −45° C. for an additional 30 min and then at 0° C. for 2 h. Di-tert-butyl dicarbonate (5.92 mL of a 1 M solution in THF, 5.92 mmol) was added to the reaction mixture at 0° C. The reaction was slowly warmed to room temperature and stirred for 6 h. The reaction was quenched with 150 mL ice water, and the organics were extracted with ethyl acetate (60 mL×2). The organic layer was then washed with brine, dried with Na₂SO₄ and concentrated. The residue was purified by flash chromatography with 0 to 12% EtOAc in hexane to give the N-Boc protected product 2 (0.66 g, 1.51 mmol, 51%) as a light-yellow solid. ¹H NMR (400 MHz, DMSO) δ 8.18 (d, J=8.9 Hz, 1H), 7.24 (t, J=8.2 Hz, 1H), 7.20 (d, J=8.9 Hz, 1H), 6.83 (dd, J=8.4, 0.7 Hz, 1H), 6.78 (dd, J=8.0, 0.9 Hz, 1H), 6.24 (s, 1H), 5.78 (s, 1H), 3.90 (s, 3H), 3.37 (s, 3H), 2.23 (s, 3H), 1.60 (s, 3H), 1.46 (s, 9H), 1.12 (s, 3H). ¹³C NMR (100 MHz, DMSO-d6) δ 157.20, 152.93, 150.68, 138.82, 138.16, 129.21, 128.21, 127.40, 126.25, 125.72, 124.81, 123.46, 112.31, 110.85, 106.52, 95.94, 81.12, 56.34, 55.16, 54.98, 28.25, 25.06, 21.73. Calculated for C26H32NO5 438.2275 (M+H)⁺ determined 438.2272.

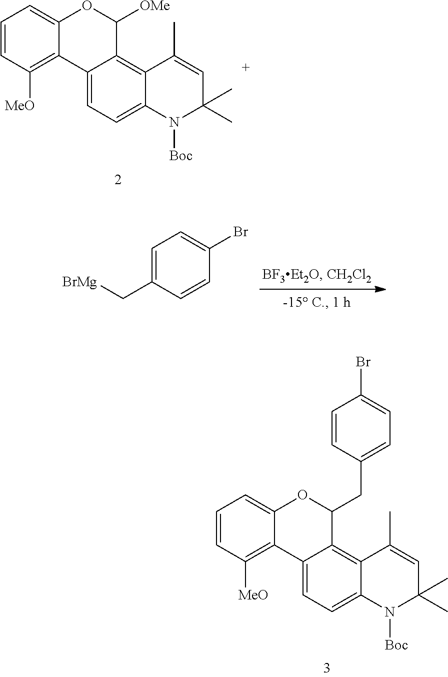

tert-Butyl-5-(4-bromobenzyl)-10-methoxy-2,2,4-trimethyl-2,5-dihydro-1H-chromeno[3,4-f]quinoline-1-carboxylate (3). A solution of 2 (200 mg, 0.457 mmol) in 15 mL of anhydrous CH₂Cl₂ was cooled to −15° C. and treated dropwise with BF₃.Et₂O (169 μL, 1.37 mmol). The mixture was stirred at −15° C. for an additional 30 min. Then the resulting deep brown solution was treated dropwise with 4-bromobenzylmagnesium bromide (10.96 mL of a 0.25 M Et₂O solution, 2.74 mmol). At the end of the addition, the brown color changed to a slightly yellow solution. After the solution was stirred for 15 min at −15° C., the reaction mixture was quenched by the addition of 10 mL of saturated aqueous NaHCO₃. The organic phase was extracted with 2×20 mL of ethyl acetate, washed with brine, dried with Na₂SO₄, filtered, and concentrated. The residue was purified by flash chromatography on silica gel with 5% ethyl acetate/hexanes to provide the compound 3 (165.6 mg, 0.288 mmol, 73%) as a light green solid. ¹H NMR (400 MHz, DMSO-d6) δ 8.23 (d, J=8.9 Hz, 1H), 7.46 (d, J=8.3 Hz, 2H), 7.24 (t, J=8.2 Hz, 1H), 7.14 (d, J=8.9 Hz, 1H), 7.05 (d, J=8.4 Hz, 2H), 6.81 (d, J=8.4 Hz, 1H), 6.52 (d, J=8.7 Hz, 1H), 5.97 (dd, J=10.2, 3.3 Hz, 1H), 5.74 (s, 1H), 3.93 (s, 3H), 3.01 (dd, J=14.9, 10.2 Hz, 1H), 2.80 (dd, J=14.9, 3.3 Hz, 1H), 2.29 (s, 3H), 1.45 (s, 9H), 1.32 (s, 6H). ¹³C NMR (100 MHz, DMSO-d6) δ 156.97, 152.47, 151.62, 139.27, 138.07, 136.88, 131.26, 131.13, 130.48, 129.22, 127.60, 125.74, 124.37, 123.59, 123.11, 119.58, 112.11, 110.54, 105.67, 80.57, 74.01, 55.84, 54.60, 36.93, 27.82, 26.12, 23.22. HR-MS: Calculated for C32H35BrNO4 576.1744 (M+H)⁺ determined 576.1740.

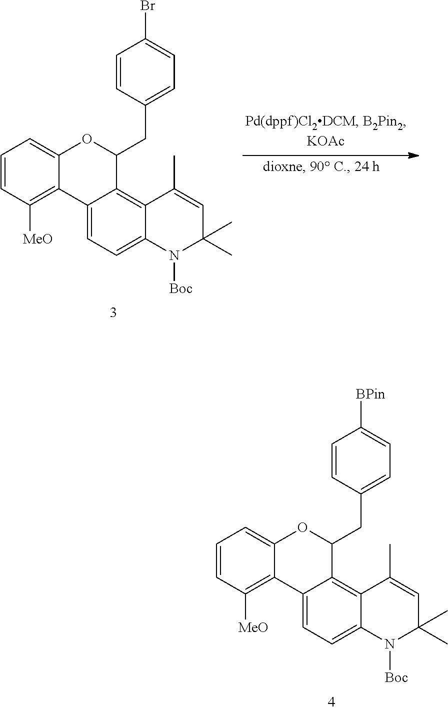

tert-Butyl-10-methoxy-2,2,4-trimethyl-5-(4-(4,4,5,5-tetramethyl-1,3,2-dioxaborolan-2-yl)benzyl)-2,5-dihydro-1H-chromeno[3,4-f]quinoline-1-carboxylate (4). Pd(dppf)Cl₂.DCM (6.1 mg, 0.0087 mmol) was added to a mixture of 3 (100 mg, 0.173 mmol), potassium acetate (49.2 mg, 0.519 mmol) and Bis(pinacolato)diboron (84.3 mg, 0.346 mmol) in anhydrous dioxane (2 mL). The mixture was stirred at 100° C. for 24 h. The insoluble solid was removed by filtration through Celite pad. The remaining solution was diluted with H₂O (30 mL) and extracted with EtOAc (3×10 mL). The combined organic extracts were washed with brine, dried over Na₂SO₄ and concentrated under vacuum. The oil was purified via flash column chromatography with 5% ethyl acetate/hexanes to obtain 4 (51.9 mg, 0.083 mmol, 68%) as an off-white solid. ¹H NMR (400 MHz, CDCl3) δ 8.31 (d, J=8.9 Hz, 1H), 7.74 (d, J=8.0 Hz, 2H), 7.23-7.13 (m, 4H), 6.67 (dd, J=8.3, 0.8 Hz, 1H), 6.57 (dd, J=8.1, 1.0 Hz, 1H), 6.05 (dd, J=10.6, 2.9 Hz, 1H), 5.67 (d, J=1.4 Hz, 1H), 3.98 (s, 3H), 3.22 (dd, J=14.9, 10.6 Hz, 1H), 2.78 (dd, J=14.9, 2.9 Hz, 1H), 2.33 (s, 3H), 1.51 (s, 9H), 1.46 (s, 3H), 1.37 (s, 3H), 1.35 (s, 12H). ¹³C NMR (100 MHz, Chloroform-d) δ 157.29, 153.41, 152.46, 141.45, 139.58, 138.51, 134.82, 131.36, 131.18, 130.93, 128.65, 128.21, 125.90, 125.04, 124.25, 123.77, 112.98, 111.44, 104.95, 83.70, 80.82, 75.14, 55.69, 55.18, 38.69, 28.30, 26.94, 24.93, 23.79. Calculated for C38H47BNO6: 624.3491 (M+H)⁺ determined 624.3491.

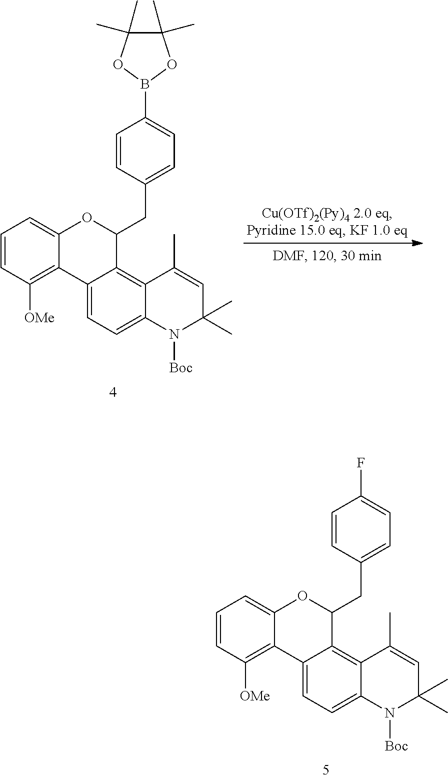 [SMILES: COc1cccc2c1-c1ccc3c(c1C(Cc1ccc(B4OC(C)(C)C(C)(C)O4)cc1)O2)C(C)=CC(C)(C)N3C(=O)OC(C)(C)C ; COc1cccc2c1-c1ccc3c(c1C(Cc1ccc(F)cc1)O2)C(C)=CC(C)(C)N3C(=O)OC(C)(C)C]

tert-Butyl-5-(4-fluorobenzyl)-10-methoxy-2,2,4-trimethyl-2,5-dihydro-1H-chromeno[3,4-f]quinoline-1-carboxylate (5). Arylboronic acid pinacol ester 4 (50 mg, 0.08 mmol) was combined with Cu(OTf)₂(Py)₄ (54 mg, 5 equiv), KF (25 mg, 5 equiv), and 18-crown-6 (151 mg, 5 equiv). DMF (2.0 mL) and pyridine (200 μL, 30 equiv) were then added to the vial. The reaction mixture stirred at 120° C. for 12 h. The organic solvent was removed under reduced pressure to give solid residue, which was purified by silica gel column chromatography (EtOAc/Hexanes) to obtain 5 (24.8 mg), in 60% yield. ¹H NMR (400 MHz, CDCl3) δ 8.30 (d, J=8.9 Hz, 1H), 7.23-7.17 (m, 2H), 7.12-7.06 (m, 2H), 6.96 (t, J=8.7 Hz, 2H), 6.68 (d, J=8.0 Hz, 1H), 6.58 (d, J=8.0 Hz, 1H), 6.00 (dd, J=10.4, 3.1 Hz, 1H), 5.66 (d, J=1.3 Hz, 1H), 3.98 (s, 3H), 3.17 (dd, J=15.0, 10.4 Hz, 1H), 2.74 (dd, J=15.0, 3.1 Hz, 1H), 2.31 (s, 3H), 1.51 (s, 9H), 1.46 (s, 3H), 1.37 (s, 3H). ¹³C NMR (100 MHz, Chloroform-d) δ 161.63 (d, J=244.4 Hz), 157.33, 153.39, 152.38, 139.63, 138.54, 133.73 (d, J=3.1 Hz), 130.97, 130.65 (d, J=7.9 Hz), 128.72, 128.10, 125.92, 125.04, 124.28, 123.69, 115.07 (d, J=21.2 Hz), 112.98, 111.16, 105.00, 80.87, 75.24, 55.69, 55.17, 37.72, 28.30, 26.93, 23.78. ¹⁹F NMR (376 MHz, Chloroform-d) 6-116.84. Calculated for C32H35FNO4: 516.2545 (M+H)⁺ determined 516.2539.

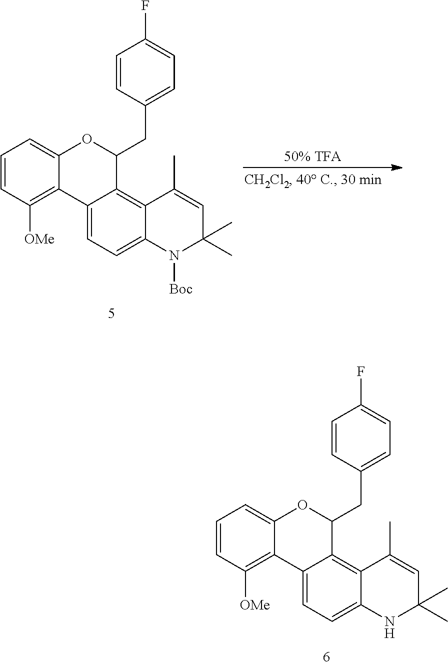 [SMILES: COc1cccc2c1-c1ccc3c(c1C(Cc1ccc(F)cc1)O2)C(C)=CC(C)(C)N3C(=O)OC(C)(C)C ; COc1cccc2c1-c1ccc3c(c1C(Cc1ccc(F)cc1)O2)C(C)=CC(C)(C)N3]

5-(4-Fluorobenzyl)-10-methoxy-2,2,4-trimethyl-2,5-dihydro-1H-chromeno[3,4-f]quinoline (6). A mixture of 5 (50 mg, 0.097 mmol) in 50% trifluoroacetic acid and DCM (1.0 ml) was stirred at 40° C. for 30 min. The mixture was then neutralized with a 5 M NaOH solution under 0° C., and the aqueous solution was extracted with EtOAc (3×5 ml). The organic layer was washed with brine (50 ml), dried over Na₂SO₄ and concentrated under vacuum. The residue was purified by silica gel chromatography (Hexane/EtOAc=95:5) to obtain 6 (38 mg, 95%) as a light-yellow solid. ¹H NMR (400 MHz, DMSO-d6) δ 8.01 (d, J=8.6 Hz, 1H), 7.13-7.07 (m, 5H), 6.74 (d, J=7.8 Hz, 1H), 6.62 (d, J=8.6 Hz, 1H), 6.46 (d, J=8.0 Hz, 1H), 6.15 (s, 1H), 5.96-5.88 (m, 1H), 5.41 (s, 1H), 3.88 (s, 3H), 2.96 (dd, J=14.8, 10.1 Hz, 1H), 2.75 (dd, J=14.8, 3.2 Hz, 1H), 2.21 (s, 3H), 1.15 (s, 3H), 1.12 (s, 3H). ¹³C NMR (100 MHz, DMSO-d6) δ 161.35 (d, J=241.8 Hz), 158.66, 156.73, 151.16, 146.07, 134.48 (d, J=3.0 Hz), 133.98, 132.04, 131.22 (d, J=8.0 Hz), 127.83, 127.60, 119.28, 116.79, 116.52, 115.35 (d, J=21.0 Hz), 113.85, 110.89, 105.96, 74.96, 56.12, 50.13, 37.60, 29.46, 24.74. ¹⁹F NMR (376 MHz, DMSO-d₆) δ −116.88 (m, 1F). Calculated for C27H26O2NF: 415.1942 (M)±determined 415.1939.

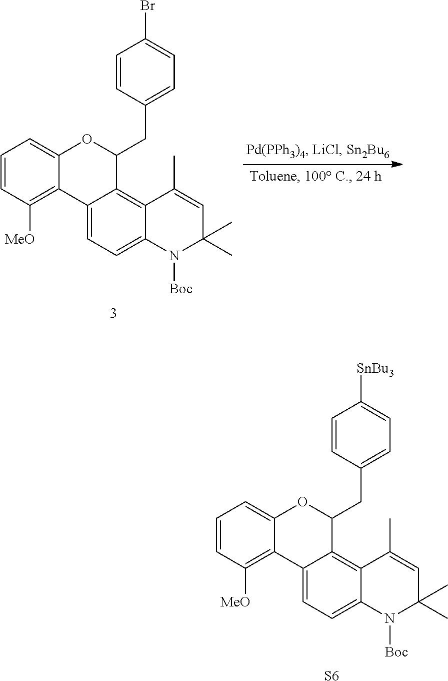

tert-Butyl-10-methoxy-2,2,4-trimethyl-5-(4-(tributylstannyl)benzyl)-2,5-dihydro-1H-chromeno[3,4-f]quinoline-1-carboxylate (S6). Aryl bromide 3 (100 mg, 0.173 mmol), was added with Pd(PPh₃)₄ (39.2 mg, 0.035 mmol), lithium chloride (35.1 mg, 0.83 mmol) to a reaction vial containing anhydrous toluene (2 mL) at room temperature. Hexabutylditin (432 μL, 0.865 mmol) was then added to the reaction via syringe, and the vial was sealed and heated to 100° C. in an oil bath. Once the reaction mixture turned black (generally 4 h), it was cooled to room temperature. Aqueous potassium fluoride (5.0 mL, 2 M solution) was then added, and the mixture was stirred vigorously for 30 min. The mixture was then filtered through a plug of Celite and washed with toluene. The filtrate was washed with brine, dried over Na₂SO₄, filtered, and concentrated under vacuum. The crude product was purified via flash column chromatography with 5% ethyl acetate/hexanes to provide the desired compound S6 (49.8 mg, 0.1 mmol, 58%) as an off-white solid. ¹H NMR (400 MHz, Chloroform-d) δ 8.31 (d, J=8.9 Hz, 1H), 7.37 (d, J=7.8 Hz, 2H), 7.20 (t, J=8.9 Hz, 2H), 7.11 (d, J=7.8 Hz, 2H), 6.67 (d, J=8.3 Hz, 1H), 6.62 (d, J=8.1 Hz, 1H), 6.04 (dd, J=10.4, 3.1 Hz, 1H), 5.65 (s, 1H), 3.98 (s, 3H), 3.17 (dd, J=14.8, 10.4 Hz, 1H), 2.73 (dd, J=14.8, 3.0 Hz, 1H), 2.32 (s, 3H), 1.55 (d, J=6.1 Hz, 6H), 1.50 (s, 9H), 1.46 (s, 3H), 1.36-1.30 (m, 9H), 1.06-1.01 (m, 6H), 0.89 (s, 9H). ¹³C NMR (100 MHz, Chloroform-d) δ 157.31, 153.41, 152.57, 139.47, 139.41, 138.49, 137.74, 136.47, 131.33, 128.93, 128.66, 128.30, 125.86, 125.09, 124.23, 123.76, 113.02, 111.39, 104.92, 80.80, 75.36, 55.69, 55.16, 38.53, 29.12, 28.30, 27.42, 26.66, 23.82, 13.71, 9.57. HR-MS: Calculated for C44H61NO4Sn 787.3623 (M)⁺ determined 787.3629.

Compounds 10-14 were prepared as described above, using corresponding Grignard reagents in place of 4-bromobenzylmagnesium bromide.

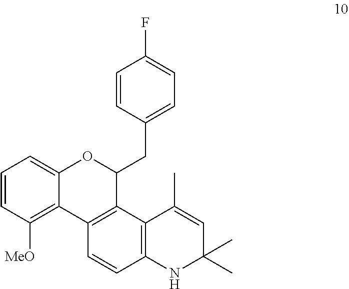 [SMILES: COc1cccc2c1-c1ccc3c(c1C(Cc1ccc(F)cc1)O2)C(C)=CC(C)(C)N3]

10-Methoxy-5-(4-fluorobenzyl)-2,5-dihydro-2,2,4-trimethyl-1H-[1]benzopyrano[3,4-f]quinoline (10): yield 90.9 mg, 0.22 mmol, 73%, as an off-white powder. ¹H NMR (400 MHz, DMSO) δ 8.03 (dd, J=8.6, 2.7 Hz, 1H), 7.14-7.06 (m, 5H), 6.74 (d, J=8.3 Hz, 1H), 6.63 (dd, J=8.6, 2.4 Hz, 1H), 6.47 (d, J=8.0 Hz, 1H), 6.13 (br, 1H), 5.93 (dd, J=10.0, 2.9 Hz, 1H), 5.41 (s, 1H), 3.88 (s, 3H), 2.97 (dd, J=14.7, 10.2 Hz, 1H), 2.75 (dd, J=14.8, 3.2 Hz, 1H), 2.21 (s, 3H), 1.16 (s, 3H), 1.13 (s, 3H). Calculated for C27H26FNO2 415.1942 (M)⁺ determined 415.1939.

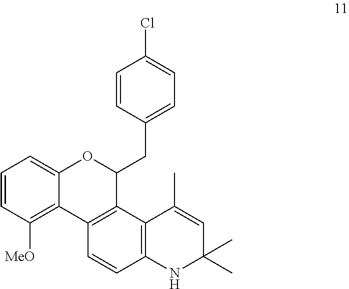 [SMILES: COc1cccc2c1-c1ccc3c(c1C(Cc1ccc(Cl)cc1)O2)C(C)=CC(C)(C)N3]

10-Methoxy-5-(4-chlorobenzyl)-2,5-dihydro-2,2,4-trimethyl-1H-[1]benzopyrano[3,4-f]quinoline (11): yield 65.9 mg, 0.15 mmol, 51%, as alight yellow powder. ¹H NMR (400 MHz, DMSO) δ 8.02 (d, J=8.6 Hz, 1H), 7.32 (d, J=8.4 Hz, 2H), 7.10 (dt, J=8.1, 3.9 Hz, 3H), 6.74 (d, J=7.8 Hz, 1H), 6.62 (d, J=8.7 Hz, 1H), 6.46 (d, J=8.7 Hz, 1H), 6.14 (s, 1H), 5.94 (dd, J=10.0, 3.4 Hz, 1H), 5.41 (s, 1H), 3.89 (s, 3H), 2.97 (dd, J=14.8, 10.0 Hz, 1H), 2.76 (dd, J=14.8, 3.3 Hz, 1H), 2.21 (s, 3H), 1.16 (s, 3H), 1.12 (s, 3H). Calculated for C27H27ClNO2 432.1725 (M+H)⁺ determined 432.1725.

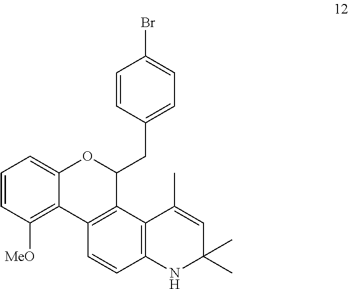 [SMILES: COc1cccc2c1-c1ccc3c(c1C(Cc1ccc(Br)cc1)O2)C(C)=CC(C)(C)N3]

10-Methoxy-5-(4-bromobenzyl)-2,5-dihydro-2,2,4-trimethyl-1H-[1]benzopyrano[3,4-f]quinoline (12): yield 89.7 mg, 0.19 mmol, 63%, as a light yellow powder. ¹H NMR (400 MHz, DMSO) δ 8.01 (d, J=8.6 Hz, 1H), 7.45 (d, J=8.3 Hz, 2H), 7.11 (t, J=8.2 Hz, 1H), 7.04 (d, J=8.4 Hz, 2H), 6.74 (d, J=7.7 Hz, 1H), 6.62 (d, J=8.6 Hz, 1H), 6.46 (d, J=7.2 Hz, 1H), 6.14 (s, 1H), 5.93 (dd, J=10.0, 3.4 Hz, 1H), 5.41 (s, 1H), 3.89 (s, 3H), 2.95 (dd, J=14.8, 10.1 Hz, 1H), 2.74 (dd, J=14.8, 3.4 Hz, 1H), 2.21 (s, 3H), 1.16 (s, 3H), 1.12 (s, 3H). Calculated for C27H26BrNO2 475.1141 (M)⁺ determined 475.1137.

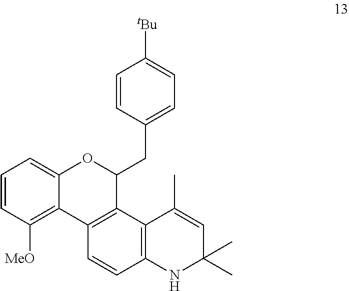 [SMILES: COc1cccc2c1-c1ccc3c(c1C(Cc1ccc(C(C)(C)C)cc1)O2)C(C)=CC(C)(C)N3]

10-Methoxy-5-(4-(tert-butyl)benzyl)-2,5-dihydro-2,2,4-trimethyl-1H-[1]benzopyrano[3,4-f]quinoline (13): yield 95.1 mg, 0.21 mmol, 70%, as an off-white powder. ¹H NMR (400 MHz, DMSO) δ 8.02 (d, J=8.6 Hz, 1H), 7.29 (d, J=8.3 Hz, 2H), 7.11 (d, J=8.1 Hz, 1H), 7.03 (d, J=8.2 Hz, 2H), 6.74 (d, J=7.8 Hz, 1H), 6.62 (d, J=8.6 Hz, 1H), 6.49 (d, J=7.3 Hz, 1H), 6.13 (s, 1H), 5.91 (dd, J=10.2, 2.9 Hz, 1H), 5.42 (s, 1H), 3.89 (s, 3H), 2.95 (dd, J=14.8, 10.3 Hz, 1H), 2.67 (dd, J=14.8, 2.8 Hz, 1H), 2.22 (s, 3H), 1.26 (s, 9H), 1.17 (s, 3H), 1.14 (s, 3H). Calculated for C31H36NO2 454.2741 (M+H)⁺ determined 454.2745.

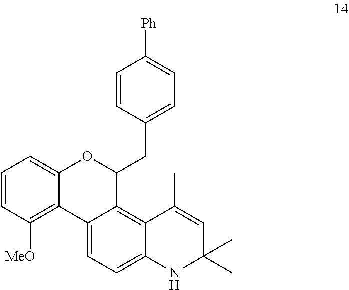 [SMILES: COc1cccc2c1-c1ccc3c(c1C(Cc1ccc(-c4ccccc4)cc1)O2)C(C)=CC(C)(C)N3]

10-Methoxy-5-(4-phenylbenzyl)-2,5-dihydro-2,2,4-trimethyl-1H-[1]benzopyrano[3,4-f]quinoline (14): yield 108 mg, 0.23 mmol, 78%, as an off-white powder. ¹H NMR (400 MHz, DMSO) δ 8.03 (d, J=8.6 Hz, 1H), 7.64 (d, J=7.2 Hz, 2H), 7.57 (d, J=8.2 Hz, 2H), 7.44 (d, J=7.8 Hz, 2H), 7.36 (d, J=7.4 Hz, 1H), 7.19 (d, J=8.2 Hz, 2H), 7.13 (t, J=8.1 Hz, 1H), 6.76 (d, J=7.7 Hz, 1H), 6.63 (d, J=8.6 Hz, 1H), 6.51 (d, J=8.8 Hz, 1H), 6.14 (s, 1H), 5.98 (dd, J=10.1, 3.1 Hz, 1H), 5.43 (s, 1H), 3.90 (s, 3H), 3.12-2.91 (m, 1H), 2.79 (dd, J=14.8, 3.1 Hz, 1H), 2.26 (s, 3H), 1.16 (s, 3H), 1.13 (s, 3H). Calculated for C33H32NO2 474.2428 (M+H)⁺ determined 474.2426.

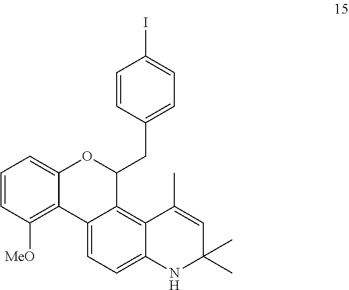 [SMILES: COc1cccc2c1-c1ccc3c(c1C(Cc1ccc(I)cc1)O2)C(C)=CC(C)(C)N3]

Compound 15 was prepared via iodination of compound 12.

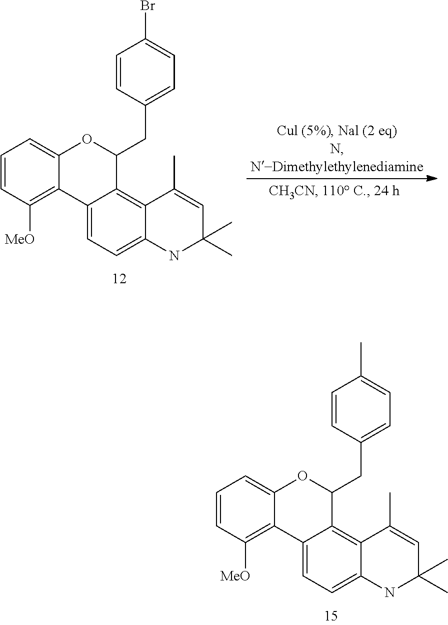 [SMILES: COc1cccc2c1-c1ccc3c(c1C(Cc1ccc(Br)cc1)O2)C(C)=CC(C)(C)N3 ; COc1cccc2c1-c1ccc3c(c1C(Cc1ccc(C)cc1)O2)C(C)=CC(C)(C)N3]

A screw-top vial (15 mL) sealed with a septum was charged with CuI (4.8 mg, 5 mol %), N,N′-dimethylenediamine (10 mol %), NaI (150 mg, 1.0 mmol) and 12 (237 mg, 0.5 mmol) in glove box followed by addition of anhydrous acetonitrile (2.0 mL). The reaction was then stirred at 110° C. in an oil bath for 24 h, cooled to r.t., quenched by the addition of 5 mL of H₂O followed by 10 mL of EtOAc and the layers separated. The aqueous phase was extracted with EtOAc, and the combined organic phases were washed with brine and dried over Na₂SO₄. The residue was purified by silica gel chromatography eluting with 5-10% EtOAc in hexanes to give the iodide 15 (230.1 mg, 1.84 mmol, 88%) as an off-white powder. ¹H NMR (400 MHz, DMSO) δ 8.01 (d, J=8.6 Hz, 1H), 7.61 (d, J=8.2 Hz, 2H), 7.10 (t, J=8.1 Hz, 1H), 6.90 (d, J=8.2 Hz, 2H), 6.74 (d, J=8.3 Hz, 1H), 6.61 (d, J=8.7 Hz, 1H), 6.45 (d, J=8.0 Hz, 1H), 6.13 (s, 1H), 5.92 (dd, J=10.0, 3.0 Hz, 1H), 5.41 (s, 1H), 3.89 (s, 3H), 2.93 (dd, J=14.7, 10.1 Hz, 1H), 2.72 (dd, J=14.8, 3.1 Hz, 1H), 2.20 (s, 3H), 1.16 (s, 3H), 1.12 (s, 3H). Calculated for C27H26INO2 523.1003 (M)⁺ determined 523.0999.

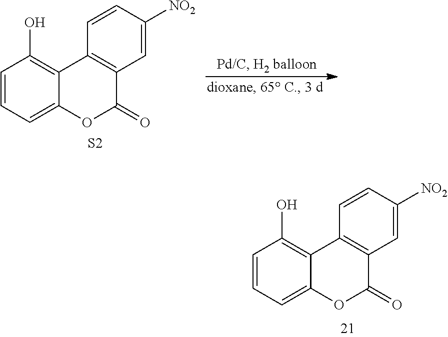

1-hydroxy-8-amino-6H-dibenzo[b,d]pyran-6-one (21); To A suspension of S2 (10.4 g, 40.5 mmol) in dry dioxane (400 mL) at r.t. was added 10% palladium on carbon (430 mg) and the suspension mixture was heated at 65° C., treated with hydrogen using balloon. After 3 d, the mixture was vacuum filtered through Celite while still hot; the filter pad was washed with hot dioxane (3×30 mL). The filtrate was concentrated and microcrystalline aniline precipitated from solution. The desired product was vacuum filtered and dried in vacuum to give aniline 21 (8.8 g, 38.9 mmol, 96%) as a yellow solid. ¹H NMR (400 MHz, DMSO) δ 11.45 (s, 1H), 9.25 (d, J=9.1 Hz, 1H), 8.82 (s, 1H), 8.62 (dd, J=9.1, 2.5 Hz, 1H), 7.44 (t, J=8.2 Hz, 1H), 6.90 (dd, J=14.9, 8.2 Hz, 2H), 2.08 (s, 2H).

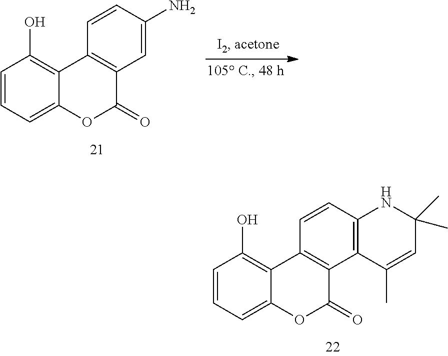 [SMILES: Nc1ccc2c(c1)c(=O)oc1cccc(O)c12 ; CC1=CC(C)(C)Nc2ccc3c(c21)c(=O)oc1cccc(O)c13]

2,5-dihydro-10-hydroxy-5-oxo-2,2,4-trimethyl-1H-[1]benzopyrano [3,4-f]quinoline (22); A solution of 21 (4.0 g, 17.6 mmol) and iodine (1.79 g, 7.04 mmol) in dry acetone (380 mL) placed into a 1 L ACE glass high pressure vessel. The reaction mixture was stirred for 48 h at 105° C., cooled to room temperature (r.t.) and concentrated. The resulting brown oil was purified by flash chromatography using silica gel with 15 to 30% EtOAc in hexanes to give the desired compound 22 (2.22 g, 7.2 mmol, 41%) as a bright yellow solid. ¹H NMR (400 MHz, DMSO) δ 10.63 (br, s, 1H), 8.78 (d, J=8.9 Hz, 1H), 7.50-6.99 (m, 2H), 6.79 (m, 3H), 5.42 (br, s, 1H), 1.93 (s, 3H), 1.22 (s, 6H).

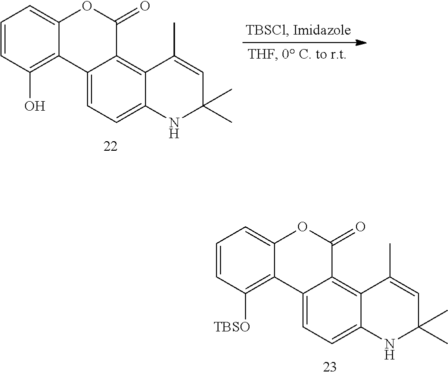 [SMILES: CC1=CC(C)(C)Nc2ccc3c(c21)c(=O)oc1cccc(O)c13 ; CC1=CC(C)(C)Nc2ccc3c(c21)c(=O)oc1cccc(O[Si](C)(C)C(C)(C)C)c13]

10-(tert-Butyldimethylsiloxy)-5-oxo-2,5-dihydro-2,2,4-trimethyl-1H-[1]benzopyrano[3,4-f]quinoline (23). A solution of phenol 22 (4.40 g, 14.4 mmol) in dry THF (200 mL) was cooled to 0° C. Then imidazole (2.93 g, 43.2 mmol) and TBSCl (3.26 g, 21.6 mmol) were added. The resulting mixture was stirred overnight at r.t. The reaction mixture was diluted with EtOAc (200 mL) and washed with 1 N HCl (3×50 mL), saturated NaHCO₃ (2×50 mL) and brine (3×50 mL). The organic layer was dried (Na₂SO₄) and concentrated. The resulting residue was purified by chromatography on silica gel with 0 to 15% EtOAc in hexanes to give the desired compound 23 (3.51 g, 8.35 mmol, 58%) as a light yellow solid. ¹H NMR (400 MHz, DMSO) δ 8.64 (d, J=8.9 Hz, 1H), 7.25 (t, J=8.2 Hz, 1H), 7.07 (d, J=9.0 Hz, 1H), 6.94 (dd, J=8.2, 0.9 Hz, 1H), 6.86 (br, s, 1H), 6.83 (dd, J=8.1, 1.0 Hz, 1H), 5.44 (br, s, 1H), 1.94 (s, 3H), 1.22 (s, 6H), 1.00 (s, 9H), 0.32 (s, 6H). HR-MS: Calculated for C25H32NO3Si 422.2146 (M+H)⁺ determined 422.2141.

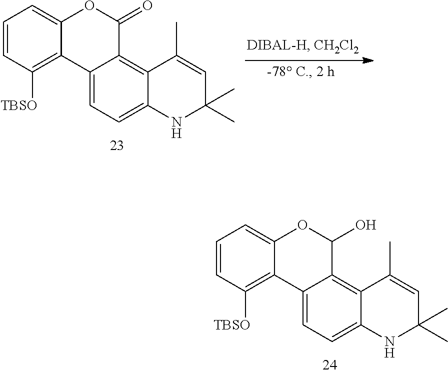 [SMILES: CC1=CC(C)(C)Nc2ccc3c(c21)c(=O)oc1cccc(O[Si](C)(C)C(C)(C)C)c13 ; CC1=CC(C)(C)Nc2ccc3c(c21)C(O)Oc1cccc(O[Si](C)(C)C(C)(C)C)c1-3]

10-(tert-butyldimethylsiloxy)-5-hydroxy-2,5-dihydro-2,2,4-trimethyl-1H-[1]benzopyrano[3,4-f]quinolone (24). Hemiacetal lactol 23 (3.51 g, 8.35 mmol) in dry CH₂Cl₂ (100 mL) was slowly added 1M Dibal-H in hexane (20.9 mL, 20.9 mmol) at −78° C. under Ar to form the orange-red solution and was stirred at −78° C. for 2 h. Then was quenched with 200 mL EtOAc and 200 mL of saturated aqueous Rochelle's salt, and mixture was stirred vigorously for 4 h at r.t. The separated aqueous layer was extracted with EtOAc, the combined organic layers were washed with brine, dried (Na₂SO₄) and concentrated to give the lactol 24 (2.83 g, 6.68 mmol, 80%) as a light yellow solid. ¹H NMR (400 MHz, DMSO) δ 7.99 (d, J=8.6 Hz, 1H), 6.98 (t, J=8.1 Hz, 1H), 6.92 (d, J=4.9 Hz, 1H), 6.69-6.47 (m, 4H), 6.08 (br, 1H), 5.41 (br, 1H), 2.23 (s, 3H), 1.26 (s, 3H), 1.08 (s, 3H), 0.97 (s, 9H), 0.25 (s, 3H), 0.10 (s, 3H). Calculated for C25H32NO3Si 422.2157 (M−H)⁻ determined 422.2147.

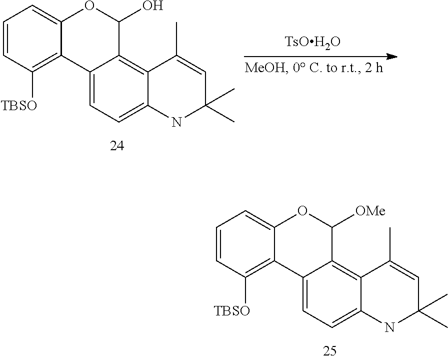 [SMILES: CC1=CC(C)(C)Nc2ccc3c(c21)C(O)Oc1cccc(O[Si](C)(C)C(C)(C)C)c1-3 ; COC1Oc2cccc(O[Si](C)(C)C(C)(C)C)c2-c2ccc3c(c21)C(C)=CC(C)(C)N3]

10-(tert-butyldimethylsiloxy)-5-methoxy-2,5-dihydro-2,2,4-trimethyl-1H-[1]benzopyrano[3,4-f]quinoline (25). The lactol 24 (2.83 g, 6.68 mmol) was dissolved in 60 mL of MeOH at 0° C., treated with the solution of p-TsOH.H₂O (550 mg) in MeOH and stirred for 30 min at 0° C. Then the cool bath was removed and stirred at r.t. for 1 h. The reaction mixture was concentrated under reduced pressure to removed most of MeOH. Then the EtOAc (200 mL) was added and the organic layers were washed with saturated aqueous NaHCO₃, brine, dried (Na₂SO₄), and concentrated. The resulting brown oil was purified by flash chromatography using silica gel with 0 to 12% EtOAc in hexane to give the desired compound 25 (2.27 g, 5.21 mmol, 78%) as an off-white powder. ¹H NMR (400 MHz, DMSO) δ 7.94 (d, J=8.5 Hz, 1H), 6.97 (t, J=8.0 Hz, 1H), 6.66 (d, J=7.8 Hz, 1H), 6.57 (m, 2H), 6.13 (m, 2H), 5.39 (br, 1H), 3.29 (s, 3H), 2.13 (s, 3H), 1.22 (s, 3H), 1.03 (s, 3H), 0.91 (s, 9H), 0.19 (s, 3H), 0.04 (s, 3H). Calculated for C26H36NO3Si 438.2459 (M+H)⁺ determined 438.2456.

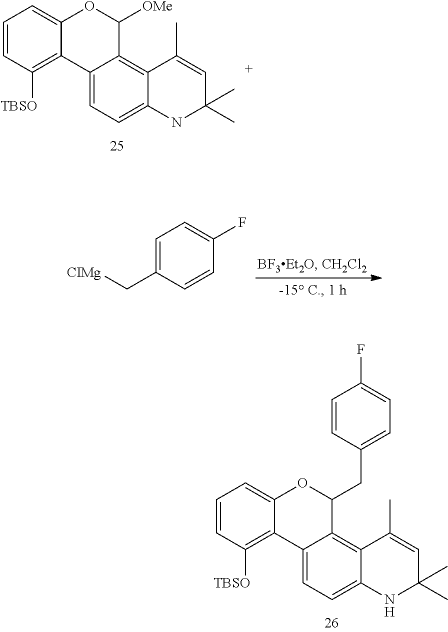

10-(tert-Butyldimethylsiloxy)-5-(4-fluorobenzyl)-2,5-dihydro-2,2,4-trimethyl-1H-[1]benzopyrano[3,4-f]quinoline (26). To a solution of 25 (437 mg, 1.00 mmol) in 10 mL dry CH₂Cl₂ was cooled to −15° C. and treated with BF₃.Et₂O (370 μL, 3.00 mmol) dropwise via syringe. The resulting deep green solution was stirred for 30 min at −15° C., treated dropwise with 4-fluorobenzylmagnesium chloride (36.0 mL of a 0.25 M in THF solution, 9.00 mmol). At the end of the addition, the green color dissipated to give a slightly yellow solution. After the solution was stirred for 15 min at −15° C., the reaction mixture was quenched by the addition of 20 mL of saturated aqueous NaHCO₃ followed by 100 mL of EtOAc and the layers separated. The aqueous phase was extracted with EtOAc, and the combined organic phases were washed with brine and dried over Na₂SO₄. The residue was purified by silica gel chromatography to give the desired compound 26 (268 mg, 0.52 mmol, 52%) as an off-white powder. ¹H NMR (400 MHz, DMSO) δ 7.97 (d, J=8.6 Hz, 1H), 7.09 (d, J=7.3 Hz, 4H), 7.03 (t, J=8.1 Hz, 1H), 6.60 (d, J=8.6 Hz, 2H), 6.48 (d, J=7.9 Hz, 1H), 6.11 (br, 1H), 5.91 (dd, J=9.7, 3.6 Hz, 1H), 5.39 (br, 1H), 2.97 (m, 1H), 2.84 (m, 1H), 2.20 (s, 3H), 1.14 (s, 6H), 0.94 (s, 9H), 0.22 (s, 3H), 0.11 (s, 3H). Calculated for C32H39FNO2Si 516.2729 (M+H)⁺ determined 516.2735.

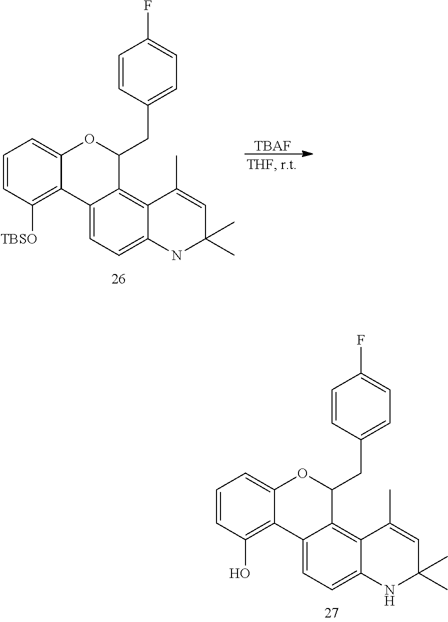 [SMILES: CC1=CC(C)(C)Nc2ccc3c(c21)C(Cc1ccc(F)cc1)Oc1cccc(O[Si](C)(C)C(C)(C)C)c1-3 ; "TBAF" ; CC1=CC(C)(C)Nc2ccc3c(c21)C(Cc1ccc(F)cc1)Oc1cccc(O)c1-3]

10-Hydroxy-5-(4-fluorobenzyl)-2,5-dihydro-2,2,4-trimethyl-1H-[1]benzopyrano[3,4-f]quinoline (27). Silyl ether 26 (1.39 g, 271 mmol) was dissolved in THF (20 mL) at r.t and was treated with TBAF (5.42 nL of a 1 M solution in THF, 5.12 mmol). After 2 h, the reaction mixture was concentrated in vacuo and was redissolved in EtOAc (60 mL). The solution was washed with brine (10 nL), then was dried (Na₂SO₄). The residue was purified by silica gel chromatography to give the desired phenol 27 (739 mg, 1.84 mmol, 68%) as an off-white powder. ¹H NMR (400 MHz, DMSO) δ 9.86 (br, 1H), 8.20 (d, J=8.6 Hz, 1H), 7.19-7.05 (m, 4H), 6.94 (t, J=8.0 Hz, 1H), 6.62 (dd, J=13.1, 8.4 Hz, 2H), 6.31 (d, J=7.8 Hz, 1H), 6.04 (br, 1H), 5.91 (d, J=7.5 Hz, 1H), 5.40 (br, 1H), 3.00 (m, 1H), 2.73 (m, 1H), 2.21 (s, 3H), 1.16 (s, 3H), 1.13 (s, 3H). Calculated for C26H25FNO2 402.1864 (M+H)⁺ determined 402.1861.

### Example 2. ¹⁸F Radiochemistry

For radiochemistry, the H¹⁸F (aq.) solution was prepared and provided by the Radiopharmaceutical core facility at UCSF. Semi-preparative reverse-phase HPLC purification of radiolabeled products was performed with a Waters 600 LC pump (Milford, Mass.) connected in series to a Shimadzu SPD-UV-Visible detector (Columbia, Md.) and a gamma counting in-line radiation flow detector (Model 105a, CRA; Berkeley, Calif.). Separations were performed on a Phenomenex Luna® C-18(2), 10 μm, 100 Å, 250×10 mm column at a 6 mL/min flow rate with UV detection at 254 nm. SRI PeakSimple software (version 304—Torrance, Calif.) was used to acquire HPLC chromatograms. QMA light cartridges for concentrating ¹⁸F-fluoride ion and C-18 light Sep-Paks were purchased from Waters Scientific (Milford, Mass.).

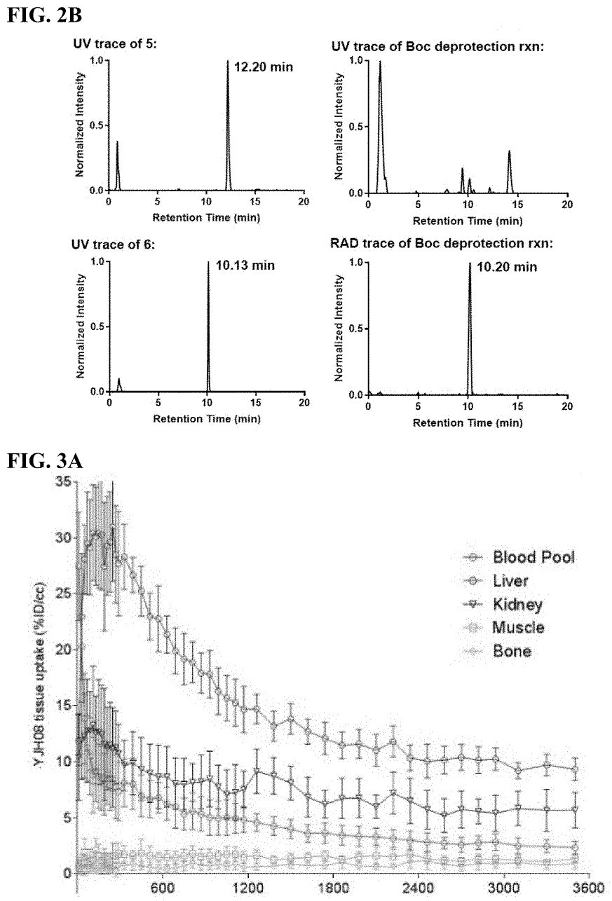

A brief screen of reaction conditions revealed that [18F]-Boc-YJH08 could be efficiently synthesized by hand in 20 min using K₂CO₃/KOTf as the base to mobilize [18F]-fluoride anion from a QMA column (4.7±0.8%). This reaction was also found to be amenable to automation using an ELIXYS FLEX/CHEM synthesis module, and HPLC analysis of the reaction progress showed that the reaction produces one major radioactive peak corresponding to [18F]-Boc-YJH08 (FIG. 2A). After purification, the Boc protecting group was removed with 50% TFA in DCM to confer [18F]-YJH08 in 12.4±3% decay corrected radiochemical yield (FIG. 2B). The molar activity of [18F]-YJH08 was calculated to be 42±1 Ci/pmol over four independent radiosyntheses.

### Example 3. ¹¹C Radiochemistry

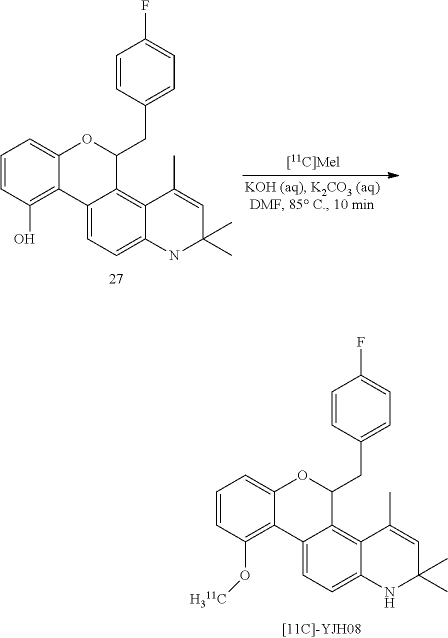 [SMILES: CC1=CC(C)(C)Nc2ccc3c(c21)C(Cc1ccc(F)cc1)Oc1cccc(O)c1-3 ; CC1=CC(C)(C)Nc2ccc3c(c21)C(Cc1ccc(F)cc1)Oc1cccc(O[11CH3])c1-3]

The radiosynthesis of [11C]-YJH08 is summarized above. Precursor 27 was pre-mixed with DMF (0.5 mL), 1.1 equiv of KOH (aq) and 20 equiv of K₂CO₃ (aq) in the 3 mL vial, and then stirred at room temperature for 10 min. In the meantime, [11C]-MeI was prepared from [11C]—CO₂ (1 Ci) in the UCSF facility. The [11C]-MeI was transferred into the reaction vail at room temperature. The reaction mixture was stirred for another 10 min at 85° C., followed by the neutralization with HCl (aq). Such reaction mixture was directly transferred from the reaction vial onto a semipreparative HPLC with C-18 column for the purification (acetonitrile/H₂O as the eluents). The fraction (Rt=8.6 min) with the [11C]-YJH08 was collected and further confirmed in the analytical HPLC (80:20 acetonitrile/water containing 0.1% formic acid, 1 ml/min); data not shown.

Radiochemical conversion (RCC) was calculated based on the radioactivity of pure product by using radio-dosimeter. 1 Ci of [11C]—CO₂ could be converted into 350 mCi of [11C]-MeI, which then reacted with labeling precursor 27 to provide [11C]-YJH08 (70 mCi, 50±4% decay corrected radiochemical yield). Moreover, further increasing the amount of base to 50 equiv or room temperature for the stirring did not result in higher RCC yield. On the other hand, high temperature (110° C.) also led to a lower yield, maybe due to the instability of final product at the high temperature. The radiochemical purity of the hot product in the analytical HPLC exceeded 95%, and its specific activity is 1±0.2 Ci4/μmol.

The product fraction from HPLC was diluted with water (20 ml), passed through an activated C-18 Sep-Pak cartridge, and followed by washing with water (10 ml). The hot compound was obtained by eluting the cartridge with CH₃CN (0.8 mL), which could be removed using reduced pressure under N2 atmosphere. The final compound was re-dissolved in the buffer for the further in vitro and in vivo studies.

[11C]-YJH02 was prepared in a similar fashion, as summarized in the following scheme:

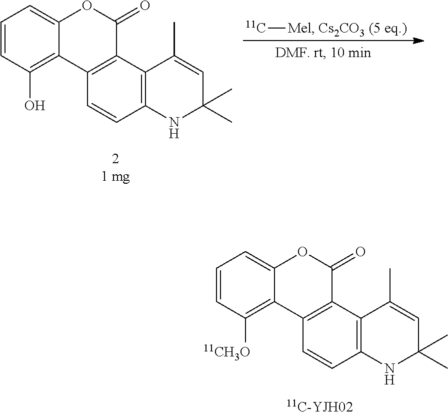

### Example 4. GR Affinity Study

The affinity of YJH08 for GR and the other type 1 subfamily 3 nuclear hormone receptors was evaluated using ³H-ligand displacement assays on cells. The K_{d} for subfamily 3 class C nuclear hormone receptors was determined on cells by displacing ³H-steroidal agonists for the respective receptor. Experimental ligands and the “reference” compounds (dexamethasone for GR, dihydrotestosterone for AR, progesterone for PR, 4-OH-tamoxifen for ER, and eplerenone for MR) were added to cells over a concentration range of 10 μM to 1 μM. Each ³H-radioligand (dexamethasone for GR, dihydrotestosterone for AR, aldosterone for MR, progesterone for PR, and estradiol for ER) was added at a molar concentration corresponding to 10× the K_{d} of the radioligand for the respective receptor. The K_{d} was determined using the following cell lines: DU145 (GR), LNCaP-AR (AR), MCF7 (PR, ER), and HEK293 transiently overexpressing full length human MR.

The cold ligands and ³H-steroids were co-incubated on cells in PBS at room temperature for one hour. Following incubation, the cells were washed twice with ice cold PBS and the unbound activity was retained for analysis. The cells were lysed with 1 mL of 1M NaOH and collected. Bound and unbound fractions were counted in a liquid scintillation counter and expressed as a percentage of the total activity added per equal relative number of cells. To determination of non-specific binding, separate treatment arms were established in which excess (>1000×K_{d}) cold reference compound was co-incubated with the experimental ligand and the ³H-steroid. These experiments were performed at three concentrations of experimental ligand (1 pM, 100 nM and 10 μM), and a linear extrapolation was used to subtract the non-specific component of binding from each treatment condition. The specific binding component was plotted against the log of the competing ligand and curve fit using a non-linear regression algorithm with PRISM software.

The K_{d} of YJH08 for GR was determined to be ˜0.4 nM, or about 10 fold more potent than the agonist dexamethasone (Table 1). Table 1 shows a summary of the binding affinity of YJH08 compared to conventional agonists for subfamily 3 nuclear hormone receptors. Reference ligands were dexamethasone (GR), dihydrotestosterone (AR), progesterone (PR), estradiol (ER), and aldosterone (MR). Abbreviations: GR=glucocorticoid receptor, AR=androgen receptor, PR=progesterone receptor, ER=estrogen receptor, MR=mineralcorticoid receptor. Moreover, YJH08 bound to GR with at least 100 fold higher relative affinity than other type 1 subfamily 3 nuclear hormone receptors.

TABLE 1

| | K_{d} (M) | | | | |
|---|---|---|---|---|---|
| Ligand | GR | AR | PR | ER | MR |
| Reference | 2.03 × | 4.96 × | 6.27 × | 6.94 × | 1.41 × |
| | 10⁻⁹ | 10⁻¹⁰ | 10⁻¹⁰ | 10⁻⁹ | 10⁻⁸ |
| YJH08 | 4.01 × | 2.20 × | 5.55 × | 1.90 × | 2.90 × |
| | 10⁻¹⁰ | 10⁻⁷ | 10⁻⁷ | 10⁻⁵ | 10⁻⁷ |

### Example 5. [18F]-YJH08 Specifically Binds GR In Vivo, and Measures Receptor Saturation by a Synthetic GR Agonist

All animal experiments were conducted under the approval from Institutional Animal Care and Use Committee (IACUC) at UCSF. Male nu nu or C57BL6/J mice (4-6 weeks) purchased from Charles River. All the mice were well-housed in the USCF with free access to food and water. For dexamethasone treatment studies, mice were treated via oral gavage with vehicle (0.5% hydroxy-propyl-methylcellulose and 0.2% Tween 80 in water) or Dexamethasone (100 mg/kg) suspended in HPMT. The mice were treated once daily for three days. At day 3, the mice received the radiotracer after the final gavage. Adrenalectomized mice were purchased from Charles River, and provided drinking water supplemented with NaCl (aq) per instructions.

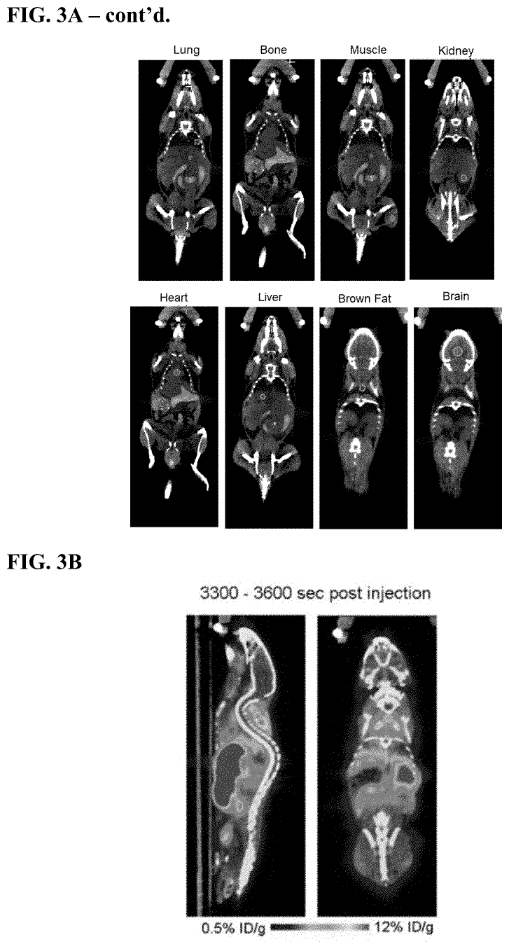

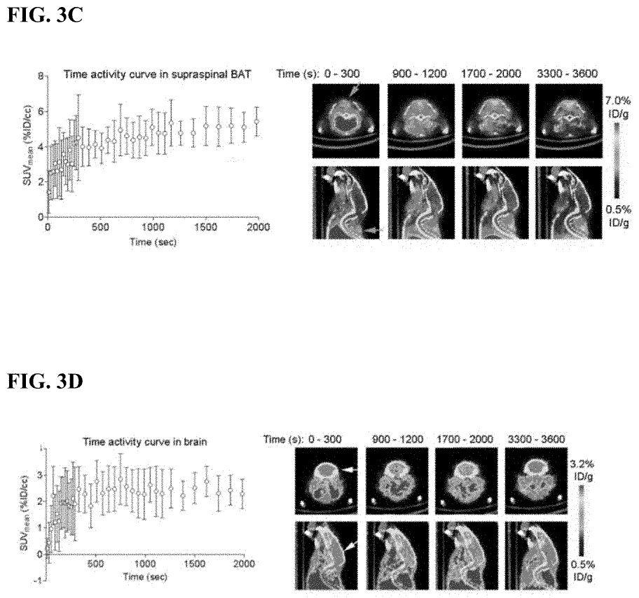

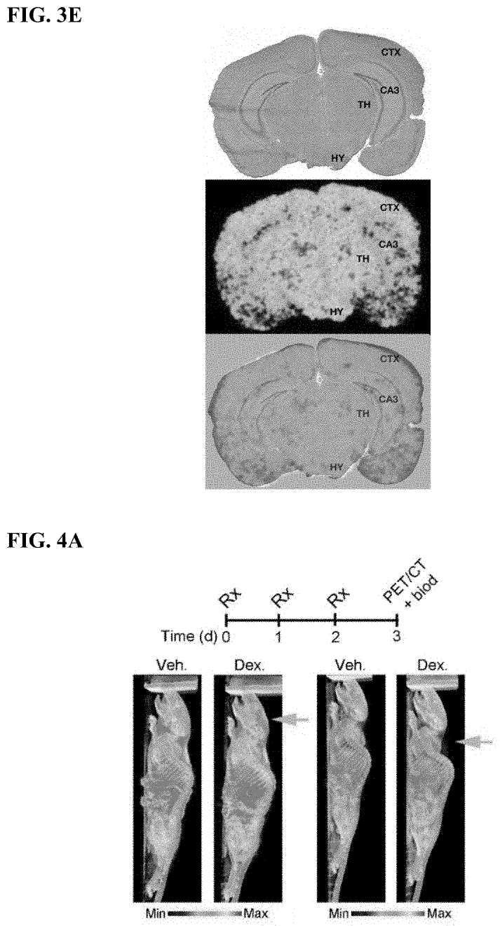

To understand the biodistribution of [18F]-YJH08 in vivo, a dynamic PET scan over 60 min was first conducted in immunocompetent male C57B16/J mice (FIG. 3A). Prior to injection, the formulated dose of [18F]-YJH08 was passed through a sterile 0.22 μm membrane. Approximately 300 μCi was injected via tail vein in a volume of 100-150 μL per mouse. After a given period of uptake time, the mice were anesthetized with 2-3% isoflurane, and imaged with a Siemens Inveon micro PET/CT. For dynamic acquisitions, the mice were anesthetized prior to injection, and injected while positioned on the scanner bed. All imaging data was decay corrected, reconstructed, and analyzed with AMIDE software. Maximum intensity projections (MIPs) were generated by AMIDE software. Regions of interest (ROI) were manually placed to calculate SUV data from the dynamic acquisitions.

As shown in FIG. 3A, the radiotracer rapidly targeted the liver within 200 sec post injection, with continuous washout from 300-3000 sec, suggesting a hepatobiliary clearance mechanism. An early increase and washout in kidney uptake was followed by stable accumulation. Muscle and bone uptake were significantly lower than liver and kidney, as expected.

The time activity curves generated using region of interest analysis revealed several enlightening trends. First, the radiotracer rapidly cleared from serum, and fitting the data with a two phase exponential decay curve estimated the fast and slow half-lives to be ˜21 sec and ˜760 sec, respectively. Moreover, liver uptake was prominent at very early time points post injection (0-300 sec), with continuous washout from ˜300-3600 sec. These data suggest a hepatobiliary mechanism of clearance. Consistent with this model, significantly lower radiotracer accumulation was observed in the kidneys from 0-3600 sec. Radiotracer uptake in the bone was low and approximately equivalent to muscle, which suggests that the compound is stable to radiodefluorination in vivo.

Remarkably, clear radiotracer uptake was also observed in tissues for which aberrant GR expression is thought to drive disease pathobiology. For instance, high uptake of the radiotracer was observed in supraspinal brown adipose tissue, a tissue in which GR expression is hypothesized to regulate seminal processes like thermogenesis and beiging. See, Lee, et al. Nuclear receptor research 2018, 5. As shown in FIG. 3C, the magnitude of radiotracer uptake was statistically higher that muscle and bone from ˜300-3600 sec post injection. At right are shown representative transaxial and sagittal images centered on the supraspinal pad of brown adipose tissue. Images from the indicated time intervals are shown to depict the accumulation of the radiotracer over time. Washout of the radiotracer from the blood pool can also be observed in the transaxial view.

Radiotracer uptake was also very high in the brain, a significant finding as the neuroimaging community has long sought to develop a non-invasive tool to measure changes in GR expression thought to be occurring in the brains of individuals suffering from mood disorders (e.g., anxiety, depression). See, Anacker, et al. Psychoneuroendocrinology 2011, 36 (3), 415-25

To better appreciate radiotracer distribution within the brain, digital autoradiography was performed. The intact C57BL6/J mouse was anesthetized and perfusion was performed with cold PBS at 20 min post-injection of the radiotracer. Tissues were collected and flash frozen in OCT on dry ice. The tissue was sectioned on a microtome at a thickness of 20 μm and immediately mounted on glass slides. The slides were then exposed on a GE phosphor storage screen, and the screen was developed on an Amersham Typhoon 9400 phosphorimager. H&E staining was performed by the Pathology core facility at UCSF. The autoradiography images were processed using ImageJ software, and aligned with high resolution images of the tissue architecture with Adobe Photoshop C6. Transverse sections showed that [18F]-YJH08 uptake was high in the hypothalamus and thalamus, with comparatively lower uptake in the CA3 region of the hippocampus (FIGS. 3D, 3E). Representative transaxial and sagittal images centered on the mouse brain (arrow) are shown at right in FIG. 3D to highlight the uptake of the radiotracer over time.

For a broader survey of radiotracer biodistribution post mortem, mice were euthanized after radiotracer injection with CO₂(g) asphyxiation and dissected at dedicated time points post injection. The blood and tissues were removed, washed, dried and weighed. The activity of each tissue was measured with a gamma counter. All data was decay corrected. PRISM software was used to express a percentage (% ID/g) of the injected dose per gram of tissue. The survey showed that several other GR rich tissue had high radiotracer uptake, including the adrenals, lungs, and pancreas (Table 2). Table 2 provides a tabular representation of the biodistribution data collected 30, 60, 90 minutes after injection of [18F]-YJH08 in C57B16/J mice. The data are represented as mean standard deviation of the % ID/g.

TABLE 2

| Tissue | 30 min | 60 min | 90 min |
|---|---|---|---|
| Blood | 0.85 ± 0.1 | 0.57 ± 0.02 | 0.49 ± 0.06 |
| Brain | 1.68 ± 0.14 | 1.33 ± 0.06 | 0.97 ± 0.03 |
| Heart | 3.04 ± 0.75 | 1.33 ± 0.21 | 0.92 ± 0.14 |
| Lung | 2.65 ± 0.19 | 2.02 ± 0.29 | 1.39 ± 0.1 |
| Liver | 11.03 ± 0.89 | 7.7 ± 0.69 | 8.43 ± 1.38 |
| Spleen | 1.59 ± 0.13 | 0.83 ± 0.02 | 0.74 ± 0.15 |
| Pancreas | 4.5 ± 0.15 | 2.63 ± 0.05 | 1.9 ± 0.41 |
| Kidney | 4.5 ± 0.5 | 2.35 ± 0.34 | 1.86 ± 0.26 |
| Stomach | 1.88 ± 0.96 | 1.63 ± 0.92 | 2.61 ± 1.54 |
| Small | 19.05 ± 3.27 | 8.61 ± 2.68 | 7.67 ± 2.34 |
| intestine | | | |
| Large | 1.33 ± 0.35 | 1.23 ± 0.43 | 6.67 ± 4.73 |
| intestine | | | |
| Supraspinal | 14.41 ± 3.42 | 16.4 ± 4.93 | 14.58 ± 1.91 |
| BAT | | | |
| Muscle | 1.49 ± 0.2 | 1.15 ± 0.03 | 0.85 ± 0.18 |
| Bone | 1.11 ± 0.15 | 0.61 ± 0.15 | 0.58 ± 0.16 |
| Adrenal | 20.02 ± 2.21 | 9.43 ± 0.08 | 5.72 ± 2.32 |
| Gland | | | |

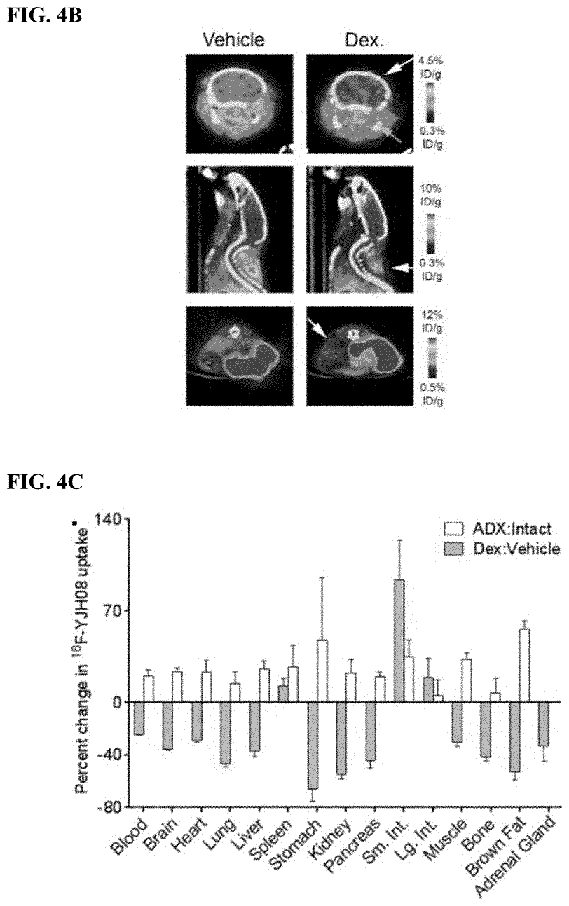

Studies were then conducted to demonstrate that [18F]-YJH08 accumulation in tissues is due to specific GR binding. First, wild type C57B16/J mice were treated with vehicle or the synthetic agonist dexamethasone via oral gavage for three days to occupy the LBD on GR. Dexamethasone treatment clearly suppressed radiotracer uptake on PET in the brain, supraspinal brown adipose tissue, and kidneys (FIGS. 4A and 4B). The images in FIG. 4A, while not strictly quantitative, are derived from equally scaled decay corrected imaging data, and capture global qualitative changes in the uptake of the radiotracer within the brain (left orange arrow) and the supraspinal brown fat pad (right orange arrow). Ex vivo biodistribution data showed that [18F]-YJH08 was significantly lower in virtually all tissues from dexamethasone treated mice compared to animals treated with vehicle (FIG. 4C and Table 3). As the graph in FIG. 4C shows, dexamethasone treatment generally suppresses radiotracer uptake in tissues due to competitive GR binding. Moreover, depletion of circulating glucocorticoids due to adrenalectomy significantly increases radiotracer uptake in all tissues assayed. The increase in radiotracer accumulation in the small intestine due to dexamethasone treatment is intriguing; it is believed that this is due to clearance to accommodate radiotracer displacement from other tissues.

TABLE 3

| Tissue | Vehicle | Dex | P value |
|---|---|---|---|
| Blood | 3.98 ± 0.08 | 2.95 ± 0.2 | 0.0002 |
| Brain | 2.05 ± 0.03 | 1.31 ± 0.07 | 0.0000 |
| Heart | 10.66 ± 0.4 | 7.57 ± 0.9 | 0.0008 |
| Lung | 6.26 ± 0.4 | 3.92 ± 0.3 | 0.0001 |
| Liver | 18.46 ± 2.9 | 11.43 ± 2.1 | 0.0081 |
| Spleen | 3.79 ± 0.4 | 4.23 ± 0.3 | 0.1295 |
| Pancreas | 6.22 ± 1.6 | 3.33 ± 0.1 | 0.0136 |
| Kidney | 8.49 ± 1.3 | 3.75 ± 0.5 | 0.0005 |
| Stomach | 1.74 ± 0.8 | 0.48 ± 0.4 | 0.0332 |
| Small | 19.12 ± 5.7 | 34.43 ± 7.2 | 0.0162 |
| intestine | | | |
| Large | 1.57 ± 0.4 | 1.77 ± 0.5 | 0.6105 |
| intestine | | | |
| Supraspinal | 10.52 ± 2.9 | 4.69 ± 0.7 | 0.0871 |
| BAT | | | |
| Muscle | 2.46 ± 0.2 | 1.71 ± 0.1 | 0.0084 |
| Bone | 2.17 ± 0.2 | 1.25 ± 0.2 | 0.0008 |
| Adrenal | 17.29 ± 6.7 | 10.40 ± 0.7 | 0.0006 |
| Gland | | | |

Table 3 shows a tabular representation of the biodistribution data collected 20 minutes after injection of [18F]-YJH08 in the context of the dexamethasone treatment study. The data are presented as a mean±standard deviation of the % ID/g. The C57B16/J mice (n=4/group) were treated via oral gavage with either vehicle or dexamethasone (100 mg/kg) once daily for three days before the radiotracer biodistribution study. The P value indicates the result of an unpaired, two-tailed Student's t test.

To understand if corticosteroid depletion elevated radiotracer uptake in tissues, the biodistribution of [18F]-YJH08 was compared in intact and adrenalectomized (adx) mice. The radiotracer uptake was uniformly higher in the tissues of adx mice compared to intact mice (FIG. 4C and Table 4). Overall, the extent of GR specific binding in tissues observed with [18F]-YJH08 is remarkable considering the previously reported biodistribution data for radiotracers targeting GR, including non-steroidal agonists. See, Wuest, et al. Bioorganic & medicinal chemistry letters 2007, 17 (14), 4035-9. Moreover, the data suggest the PET assay is sufficiently sensitive and robust to measure GR occupancy by endogenous or synthetic ligands.

Table 4 shows a tabular representation of the biodistribution data collected 20 minutes after injection of [18F]-YJH08 in the context of the comparison between uptake in intact or adrenalectomized mice. The P value indicates the result of an unpaired, two-tailed Student's t test between the two arms (n=4/group).

TABLE 4

| Tissue | Intact | Adx | P value |
|---|---|---|---|
| Blood | 2.01 ± 0.4 | 2.67 ± 0.1 | 0.029 |
| Brain | 1.96 ± 0.09 | 2.42 ± 0.1 | 0.001 |
| Heart | 3.92 ± 0.5 | 4.75 ± 1.0 | 0.209 |
| Lung | 3.89 ± 0.7 | 4.35 ± 1.1 | 0.529 |
| Liver | 10.68 ± 1.1 | 13.28 ± 1.43 | 0.028 |
| Spleen | 2.67 ± −0.5 | 3.25 ± 0.05 | 0.098 |
| Pancreas | 4.40 ± 0.2 | 5.25 ± 0.4 | 0.017 |
| Kidney | 5.79 ± 1.0 | 6.91 ± 0.3 | 0.098 |
| Stomach | 3.38 ± 2.8 | 3.23 ± 3.57 | 0.950 |
| Small | 21.22 ± 4.1 | 27.83 ± 2.89 | 0.040 |
| intestine | | | |
| Large | 1.33 ± 0-3 | 1.34 ± 0.7 | 0.985 |
| intestine | | | |
| Supraspinal | 9.01 ± 2.0 | 15.56 ± 3.8 | 0.024 |
| BAT | | | |
| Muscle | 1.53 ± 0.1 | 2.03 ± 0.2 | 0.007 |
| Bone | 1.39 ± 02 | 1.45 ± 0.2 | 0.743 |
| Adrenal | 10.82 ± 5.9 | N/A | N/A |
| Gland | | | |

### Example 6. [18F]-YJH08 Detects Endogenous GR Expression Levels in a Cancer Expected to Harbor Heterogeneous Expression Clinically

Recent data have shown that GR overexpression in a cancer cell is an adaptive response to overcome standard of care chemotherapies in cancers as molecularly diverse as prostate, breast and ovarian. See, Kach, supra; Veneris, et al. Gynecologic oncology 2017, 146 (1), 153-160. These findings have motivated several ongoing clinical trials testing the antitumor effects of GR antagonists (e.g., CORT125281, ORIC-101, RU486) alone and in combination with chemotherapy. These trials are currently accruing without biomarkers to test for tumor expression of GR, and GR expression is known to be highly variable in these cancers. Therefore, experiments were conducted to determine if [18F]-YJH08 can detect endogenous GR overexpression in a human cancer model.

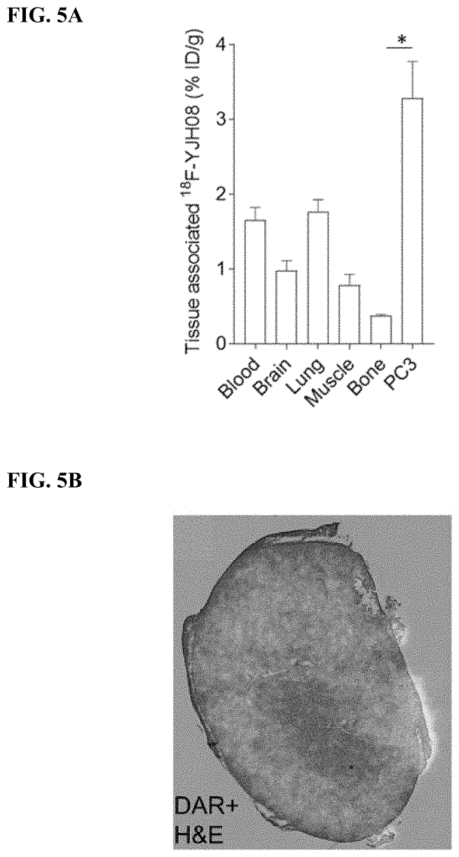

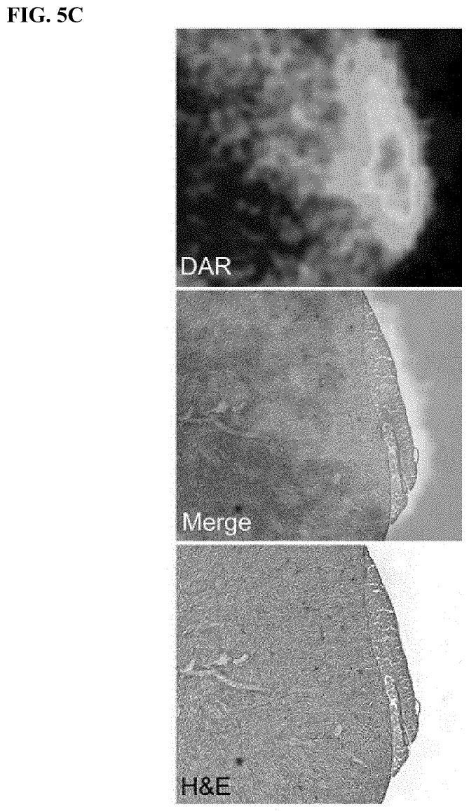

For tumor imaging studies, male nu/nu mice were used for the renal capsule tumor implants. The mice were anesthetized with isofluorane (2-3%) and performed with a dorsal midline incision (0.5 cm). With pressure applied on the muscle wall, one kidney was pulled gently and carefully through the small incision. PC3 cells (5×10 in 50 μL PBS) were injected under the kidney capsule, which was lifted from the kidney parenchyma. The kidney was placed into the mouse, and the incision closed with 3 surgical sutures. Carprofen (5-10 mg/kg) was administered to the mice during recovery. Mice were observed carefully over 24 hours for the signs of post-operative bleeding, pain and (or) other complications. After surgery, a 14 T Agilent small animal MRI was used to monitor the tumor progression. Animals were studied approximately 10 days post injection of tumor cells.

[18F]-YJH08 significantly accumulated in PC3 tumors (a human prostate cancer model) implanted in the renal capsule of mice (FIG. 5A). Radiotracer uptake (˜3% ID/g) was significantly higher in the tumor compared to other GR rich tissues like the brain and lungs. Moreover, the radiotracer uptake was significantly higher than what was observed in bone, and since bone is the most common site of prostate cancer metastasis, this finding suggests [18F]-YJH08 could be effective clinically to prospectively identify prostate cancer patients whose disease is driven by GR activity. Lastly, digital autoradiography of the affected kidney was applied to define relative uptake of the radiotracer in the tumor and normal kidney parenchyma (FIGS. 5B and 5C). The radiotracer uptake in the tumor was qualitatively much higher than the level observed in the kidney.

### Example 7. Measuring Glucocorticoid Receptor Expression In Vivo with (f)-11C-YJH08 PET

Animal Studies. All animal experiments were performed under the approval of the Institutional Animal Care and Use Committee (IACUC) at UCSF. C57BL6/J mice (4-6 weeks) were purchased from Charles River. All the mice were housed in a dedicated vivarium with free access to food and water. For dexamethasone treatment studies, dex was administered via two routes involving oral gavage of 50 mg/kg (formulation: 0.5% hydroxy-propyl-methylcellulose and 0.2% Tween 80 in water) for three days prior to radiotracer injection or administration of a water soluble dexamethasone-cyclodextrin (CD) complex via intraperitoneal injection at 10 mg/kg in PBS one hour prior to radiotracer injection. Adrenalectomized mice were purchased from Charles River and provided drinking water supplemented with NaCl (aq) per instructions until the time of the tracer biodistribution study.

Serum Stability Studies. Mice were euthanized, received radiotracer (30 MBq in 100-150 μL formulation) via tail injection, and dissected at dedicated time points post injection. The blood was collected and an aliquot (50 uL) was mixed with CH₃CN (50 uL). The serum proteins were precipitated out and followed by a centrifugation (10000 rpm for 5 min). The cleared supernatant (50 uL) was collected and further analyzed by RAD-iTLC.

Plasma Protein Binding Studies. The rapid equilibrium dialysis (RED) device inserts along with a Teflon base plate (Pierce, Rockford, Ill.) were used for the binding studies. The pH of the human or mouse plasma was adjusted to 7.4 prior to the experiment. DMSO stocks (1 mM) were spiked into the plasma to make a final concentration of 2 μM. The spiked plasma solutions (300 μL) were placed into the sample chamber (indicated by the red ring); and 500 μL of PBS buffer, pH 7.4, was placed into the adjacent chamber. The plate was incubated at 37° C. on an orbital shaker (250 rpm) for 4 hours.

After 4 hours, from the RED plate, aliquots (100 μL) were removed from each side of the insert (plasma and buffer) and dispensed into the 96-well plate. Subsequently, 100 μL of blank plasma was added to the buffer samples and 100 μL of blank buffer was added to all the collected plasma samples. 300 μL of quench solution (50% acetonitrile, 50% methanol, and 0.05% formic acid, warmed up at 37° C.) containing internal standards was added to each well. Plates were sealed, vortexed, and centrifuged at 4° C. for 15 minutes at 4000 rpm. The supernatant was transferred to fresh plates for LC/MS/MS analysis. The sample were analyzed on LC/MS/MS using an AB Sciex API 4000 instrument, coupled to a Shimadzu LC-20AD LC Pump system. Analytical samples were separated using a Waters Atlantis T3 dC18 reverse phase HPLC column (20 mm×2.1 mm) at a flow rate of 0.5 mL/min. The mobile phase consisted of 0.1% formic acid in water (solvent A) and 0.1% formic acid in acetonitrile (solvent B).

The percentage of test compound bound to protein was calculated by the following equations:

% Free=(Concentration in buffer chamber/Concentration in plasma chamber)×100%

% Bound=100%−% Free

Small animal PET/CT Imaging. Data were acquired with a Siemens Inveon microPET/CT. For dynamic acquisitions, the mice were anesthetized prior with 1.5-2% isoflurane and positioned on the scanner bed, then approximately 30 MBq of (±)-¹¹C-YJH08 was injected via tail vein in a volume of 100-150 μL per mouse. All PET imaging data were decay corrected, reconstructed with CT-based attenuation correction, and analyzed with AMIDE software. Regions of interest were manually placed to calculate SUV mean data from the dynamic acquisitions.

Biodistribution Studies. Mice were euthanized after radiotracer (7.4-18.5 MBq in 100-150 μL) injection with CO₂ (g) asphyxiation and dissected at dedicated time points post injection. The blood and tissues were collected, washed, dried, and weighed. The activity of each tissue was measured with a gamma counter. All data was decay corrected. PRISM software was used to express a percentage (% ID/g) of the injected dose per gram of tissue.

Normal Tissue Radiation Dose Estimation. Volumes of interest (VOIs) were drawn on coregistered CT images for brain, lungs, heart, liver, stomach, kidneys, and urinary bladder. All VOIs were either elliptical cylinder (5 mm long axis, 3 mm short axis, and 5 mm height for the brain), cylinders (3 mm diameter and 3 mm height for kidneys), or spheres (3 mm diameter for lungs, heart, liver, stomach, and urinary bladder), and they were placed well within the anatomical boundaries to minimize spill-over or spill-in of radioactivity. The mean values (in Bq/ml) in these VOIs were multiplied by standard mouse organ volumes (in ml) to estimate total activity (in Bq) within these organs. The total activity within the entire animal subtracted by all organ activities was used as activity in the remainder of the body. The percent of injected activity within the defined organs (% IA) was extrapolated to human-equivalent values using ratios of standard human organ weights to mouse organ weights.

These input % IA data for each organ and the remainder of the body were curve-fitted to derive time-integrated activity coefficients (also known as residence times) (in Bq-hr/Bq) and organ and whole-body effective doses for human equivalents were estimated using each mouse data. The data from the three animals were averaged to derive absorbed organs doses (in mGy/MBq) and whole-body effective dose (in mSv/MBq). Organ and effective dose estimations were performed using OLINDA version 2.0 using ICRP Publication 103 tissue weighting factors.

Autoradiography. After 20 min post-injection of (±)-¹¹C-YJH08 (˜30 MBq per mouse), mice were anesthetized and were perfused with cold PBS via cardiac puncture. Tissues were immediately collected, and flash frozen in OCT on dry ice. Tissues were sectioned on a microtome at a thickness of 20 μm and immediately mounted on glass slides. The slides were then exposed on a GE phosphor storage screen, and the screen was developed on an Amersham Typhoon 9400 phosphorimager. The autoradiography images were processed using ImageJ software.

Statistics. All statistical analysis was performed using PRISM v8.0 software. An unpaired, two-tailed Student's t test was used to determine statistically significant differences in the data. P<0.05 was reported as statistically significant. For the determination of Ki, the data were fit with a one site nonlinear regression model.

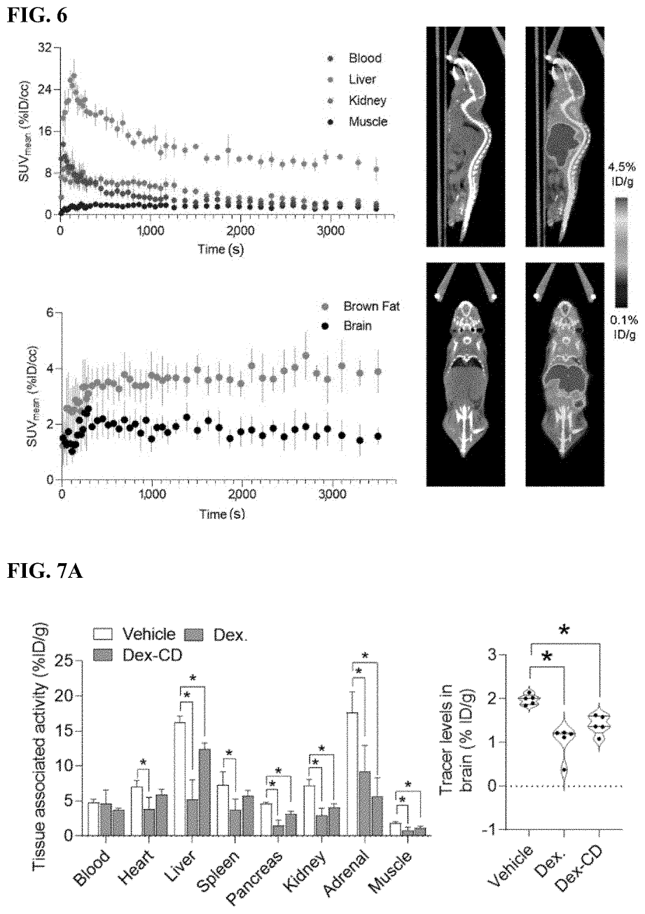

In vivo Imaging Studies with (f)-¹¹C-YJH08. A dynamic PET acquisition in C57BL6/J mice was conducted to determine the radiotracer biodistribution in vivo. Region of interest analysis of the 60 min scan showed that (±)-¹¹C-YJH08 rapidly cleared from the serum and the primary mode of clearance appeared to be hepatobiliary (FIG. 6). Moreover, tracer uptake in several organs of interest to GR biology (e.g. kidneys, brain, and adipose tissue) were above the reference compartments of blood and muscle levels at 90 min post injection and saturable over time (FIG. 6).

To confirm stability, blood was collected at 30 minutes post injection, a time point after which specific binding in vivo with (±)—¹⁸F-YJH08 occurred. Resolution of the radiolabeled metabolites on ITLC suggested the radiotracer was >90% stable at this time point (data not shown). A plasma protein binding assay was conducted to determine the percentage of free versus protein bound compound. (±)-YJH08 was almost completely bound to mouse or human serum protein (Table 5). Table 5 shows Results from duplicate plasma protein binding assays with (±)-YJH08 in mouse or human serum. Propranolol was included as a reference standard. The results in Table 5 suggest that the ligand, like endogenous corticosteroids, utilizes corticosteroid binding globulin to traffic to tissues (Henley D, et al. Pharmacol Ther. 2016; 166:128-35).

TABLE 5

| | | Concentration | % | % Bound | % |
|---|---|---|---|---|---|
| Species | Compound | (μM) | Bound | Average | Recovery |
| Human | Propranolol | 2 | 64.57 | 64.57 | 104.13 |
| | | | 64.56 | | |
| | (±)-YJH08 | 2 | >99.99 | >99.99 | 97.56 |
| | | | >99.99 | | |
| Mouse | Propranolol | 2 | 78.25 | 78.00 | 105.82 |
| | | | 77.74 | | |
| | (±)-YJH08 | 2 | >99.99 | >99.99 | 100.31 |
| | | | >99.99 | | |

Lastly, comparing the tissue uptake of carbon-11 versus fluorine-18 labeled (±)-YJH08 in C57B16/J mice at 20 min post injection showed no significant differences (Table 6). Table 6 shows a summary of the biodistribution data for (±)-¹¹C-YJH08 versus (±)-18F-YJH08. The data were collected in intact male C57B16/J mice at 20 min post injection.

TABLE 6

| | (±)-¹¹C-YJH08 | (±)-¹⁸F-YJH08 |
|---|---|---|
| Tissue | (% ID/g) | (% ID/g) |
| Blood | 2.25 ± 0.21 | 2.22 ± 0.14 |
| Brain | 1.81 ± 0.17 | 1.93 ± 0.07 |
| Heart | 4.47 ± 0.43 | 4.15 ± 0.31 |
| Liver | 12.19 ± 1.22 | 11.08 ± 0.92 |
| Spleen | 2.22 ± 0.33 | 2.46 ± 0.53 |
| Kidneys | 5.82 ± 0.28 | 5.79 ± 1.07 |
| Supraspinal | 9.8 ± 2.83 | 9.01 ± 2.07 |
| brown fat depot | | |
| Adrenal gland | 13.88 ± 7.15 | 12.19 ± 6.46 |
| Muscle | 1.53 ± 0.13 | 1.53 ± 0.12 |
| Bone | 1.19 ± 0.13 | 1.3 ± 0.23 |

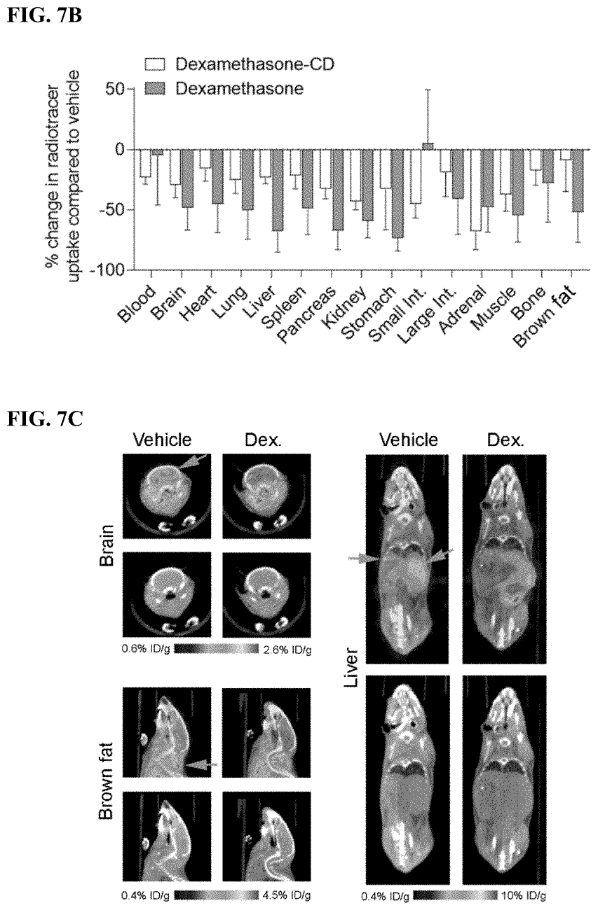

Evidence of specific binding by (±)-¹¹C-YJH08 was then studied in vivo. First, wild type C57B16/J mice were treated with dexamethasone for three days via oral gavage (50 mg/kg), an approach that has been used to saturate GR in vivo (Huang, et al. ACS Chem Biol. 2020; 15:1381-1391; Truillet C, et al. Oncotarget. 2018; 9:20399-20408). Dexamethasone treatment by this route significantly reduced (±)-¹¹C-YJH08 uptake in numerous normal tissues (FIG. 7A and Table 7).

Table 7 summarizes the biodistribution data collected 20 minutes after injection of (±)-¹¹C-YJH08 among three treatment arms receiving (1) vehicle, (2) dexamethasone at 50 mg/kg via gavage once daily for three days, and (3) one intraperitoneal injection of the water soluble dexamethasone-cyclodextrin complex at 10 mg/kg one hour prior to radiotracer administration. The P value indicates the result of an unpaired, two-tailed Student's t test between the respective treatment group and vehicle (n=5/group).

TABLE 7

| | | Dexamethasone | | Dex-CD | |
|---|---|---|---|---|---|
| | Vehicle | (50 mg/kg) | | (10 mg/kg) | |
| Tissue | Mean ± SD | Mean ± SD | P value | Mean ± SD | P value |
| Blood | 4.82 ± 0.4 | 4.58 ± 1.9 | 0.8 | 3.72 ± 0.2 | 0.001 |
| Brain | 1.98 ± 0.1 | 1.02 ± 0.3 | 0.002 | 1.40 ± 0.2 | 0.001 |
| Heart | 7.06 ± 0.8 | 3.86 ± 1.6 | 0.008 | 5.95 ± 0.7 | 0.06 |
| Lung | 6.80 ± 0.7 | 3.39 ± 1.6 | 0.006 | 5.09 ± 0.7 | 0.006 |
| Liver | 16.25 ± 0.8 | 5.22 ± 2.7 | 0.0004 | 12.47 ± 0.8 | 0.0001 |
| Spleen | 7.28 ± 1.8 | 3.71 ± 1.5 | 0.012 | 5.72 ± 0.7 | 0.14 |
| Pancreas | 4.64 ± 0.1 | 1.52 ± 0.7 | 0.0004 | 3.14 ± 0.3 | 0.0003 |
| Kidney | 7.22 ± 0.8 | 2.96 ± 0.9 | 0.0001 | 4.11 ± 0.4 | 0.0003 |
| Stomach | 2.62 ± 1.5 | 0.71 ± 0.2 | 0.04 | 1.79 ± 0.8 | 0.3 |
| Sm. Int. | 28.72 ± 14.5 | 30.24 ± 12.7 | 0.8 | 15.72 ± 3.2 | 0.1 |
| Lg. Int. | 1.70 ± 0.3 | 1.01 ± 0.5 | 0.03 | 1.38 ± 0.3 | 0.1 |
| Adrenal | 17.63 ± 2.9 | 9.24 ± 3.6 | 0.004 | 5.67 ± 2.6 | 0.0001 |
| Muscle | 1.85 ± 0.1 | 0.84 ± 0.4 | 0.003 | 1.16 ± 0.2 | 0.001 |
| Bone | 1.46 ± 0.5 | 1.06 ± 0.4 | 0.23 | 1.95 ± 1.6 | 0.5 |
| Brown Fat | 9.52 ± 0.9 | 4.58 ± 2.3 | 0.006 | 8.68 ± 2.4 | 0.5 |

Suppression of (±)-¹¹C-YJH08 binding in tissues via acute dosing with a more bioavailable formulation of dexamethasone in complex with cyclodextrin (Dex-CD) was also studied. Significant reductions in (±)-¹¹C-YJH08 tissue uptake were observed by treating mice with Dex-CD at 10 mg/kg via intraperitoneal injection 1 hour before the radiotracer injection (FIG. 7A). Notably, both blocking approaches significantly reduced tracer binding in the brain (FIG. 7A). Comparing the relative suppression of radiotracer binding across tissues showed that chronic, high doses of dexamethasone generally resulted in more significant blockade of radiotracer binding per tissue, consistent with a GR specific dose response (FIG. 7B). Visually inspecting the decay corrected PET data showed (±)-¹¹C-YJH08 suppression by dexamethasone in several tissue compartments, for example, the brain, the supraspinal depot of brown fat, and the liver (FIG. 7C). Specific binding in vivo was further studied by investigating relative tissue uptake in intact versus adrenalectomized mice (adrenalectomy depletes serum corticosteroids and elevates GR expression in tissues). As expected, tracer uptake was significantly higher in several tissues of adrenalectomized mice, including the brain Table 8). Table 8 summarizes the biodistribution data collected 20 minutes after injection of (±)-¹¹C-YJH08 in intact or adrenalectomized (ADREX) mice. The P value indicates the result of an unpaired, two-tailed Student's t test between the two arms (n=4/group).

TABLE 8

| Tissue | Intact | ADREX | P value |
|---|---|---|---|
| Blood | 2.25 ± 0.21 | 2.79 ± 0.54 | 0.105941 |
| Brain | 1.81 ± 0.17 | 2.28 ± 0.46 | 0.004892 |
| Heart | 4.47 ± 0.43 | 5.63 ± 0.94 | 0.065629 |
| Liver | 12.19 ± 1.22 | 15.21 ± 3.39 | 0.144531 |
| Spleen | 2.22 ± 0.33 | 2.88 ± 0.68 | 0.130557 |
| Kidneys | 5.82 ± 0.28 | 6.88 ± 1.52 | 0.219835 |
| Muscle | 1.53 ± 0.13 | 2.32 ± 0.5 | 0.021735 |
| Bone | 1.19 ± 0.13 | 2.03 ± 0.4 | 0.007503 |
| Supraspinal | 9.8 ± 2.83 | 12.01 ± 3.54 | 0.069191 |
| BAT | | | |
| Adrenal | 13.88 ± 7.15 | N/A | N/A |
| Gland | | | |

The relative induction of radiotracer uptake per tissue in adrenalectomized versus intact mice was qualitatively very similar between (±)-¹¹C- and ¹⁸F-YJH08 (data not shown). Lastly, the radiotracer uptake within the brain was compared using autoradiography and the overall patterns of (±)-¹¹C- and ¹⁸F-YJH08 binding were qualitatively similar between representative brain sections equidistant from bregma (data not shown).

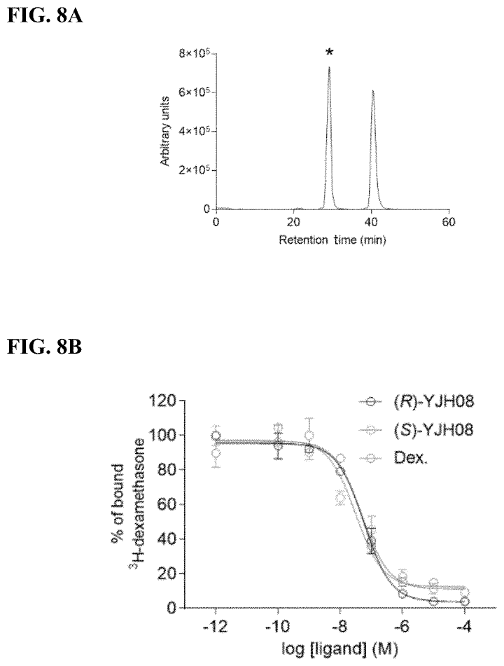

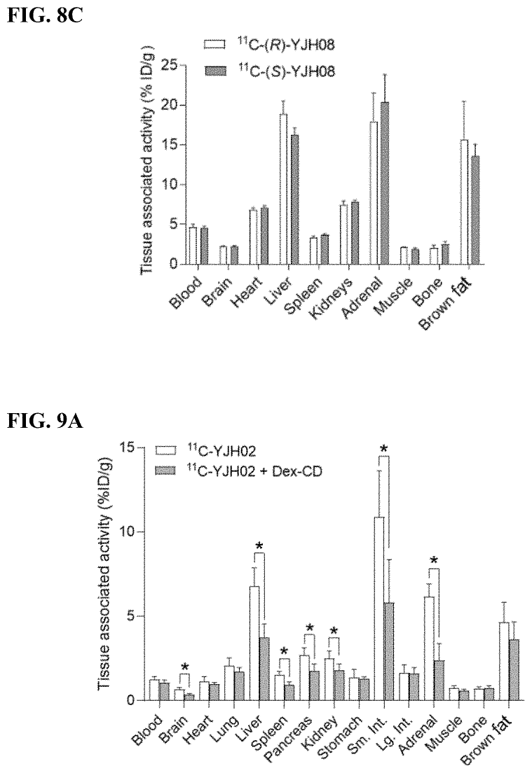

Enantiomers of YJH08 are equipotent for GR and have similar biodistribution profiles in vivo. Since YJH08 is a racemic mixture, the behavior of the (R) and (S) enantiomers was studied in vitro and in vivo, which could provide a rationale for translating enantiomerically pure YJH08. The racemic mixture of the phenol 27 was resolved using a chiral Lux® 5p Cellulose-1 stationary phase with an isocratic mobile phase of 30% water, 70% acetonitrile, and 0.1% trifluoroacetic acid (v v) (FIG. 8A). Several milligrams of one peak were isolated and the compound was recrystallized in hexanes. X ray crystallography showed the peak eluting at 29.1 min to be the (R) enantiomer (data not shown). The in vitro affinity of each enantiomer for GR in cells was tested using a ³H-dexamethasone displacement assay. (R) and (S) enantiomers had equivalent low nM affinity for GR, with Ki values of 3.76 nM (95% confidence interval=2.93-4.8) and 2.03 nM (95% confidence interval=0.78-5.3), respectively (FIG. 8B). Thus, the enantiomers are equipotent with dexamethasone and have approximately 2 fold higher affinity than endogenous corticosteroids like cortisol or corticosterone.

The (R) and (S) phenol were radiolabeled with ¹¹C-methyl iodide using the protocol outlined above. Each enantiomer labeled effectively to 55% and 48% respective decay corrected radiochemical yield from starting ¹¹C-methyl iodide (data not shown). A biodistribution study in C57B16/J mice showed that tissue uptake of both enantiomers was very similar to each other (and racemic ¹¹C-YJH08) at 20 min post injection (FIG. 8C).

These somewhat surprising results prompted a probe of the structural basis of the YJH08/GR interaction with molecular dynamics simulations. For docking studies, a co-crystal structure of the GR ligand binding domain with the corticoisteroid agonist budesonide (PDB: 5NFP) was used as a scaffold. Both enantiomers of YJH08 were prepared using the ligand preparation protocol in Maestro® and then docked into the binding site. Single precision (SP) and extra precision (XP) docking calculations were made with a flexible ligand and rigid receptor, within Maestro®'s docking suite. The resulting scores, SP: −7.3 (S), −8.1 (R); XP: −7.3 (S), −8.1 (R), were similar in value. Upon analysis of the binding pose within the site, hydrophobic interactions were observed. Collectively, these data show that the ligand binding domain on GR can accommodate both enantiomers through hydrophobic interactions.

Analysis of the Structure Activity Relationship for YJH08 Reveals the Importance of an Aryl Moiety on the C5 Position to Affinity for GR and Uptake into the Brain. To better understand the structure activity relationship between YJH08 and GR, a focused chemical screen of five YJH08 analogues bearing diverse moieties on the C5 position of the scaffold was performed. The in vitro affinities of YJH01-5 for GR were determined, showing that an aromatic group extending from the C5 position is important for a high affinity binding (Table 9). Table 9 summarizes the Ki, 95% confidence intervals, and the coefficient of determination for YJH08. The data were calculated from ³H-dexamethasone displacement assay on cells. The Ki were calculated using a one site Ki fit nonlinear regression with Prism v8.0. The data are representative of two independent assays.

TABLE 9

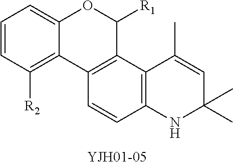

| Ligand | R₁ | R₂ | Ki (nM) | 95% CI | R² |
|---|---|---|---|---|---|
| YJH01 | ═O | OH | 2.35 | 1.15 − 4.59 | 0.96 |
| YJH02 | ═O | OMe | 2.67 | 1.32 − 5.28 | 0.93 |
| YJH03 | OH | OMe | 6.51 | 3.52 − 12.8 | 0.94 |
| YJH04 | OMe | OMe | 6.22 | 1.00 − 3.85 | 0.96 |
| YJH05 | PhCH₂ | OMe | 0.326 | 0.112 − 0.789 | 0.92 |

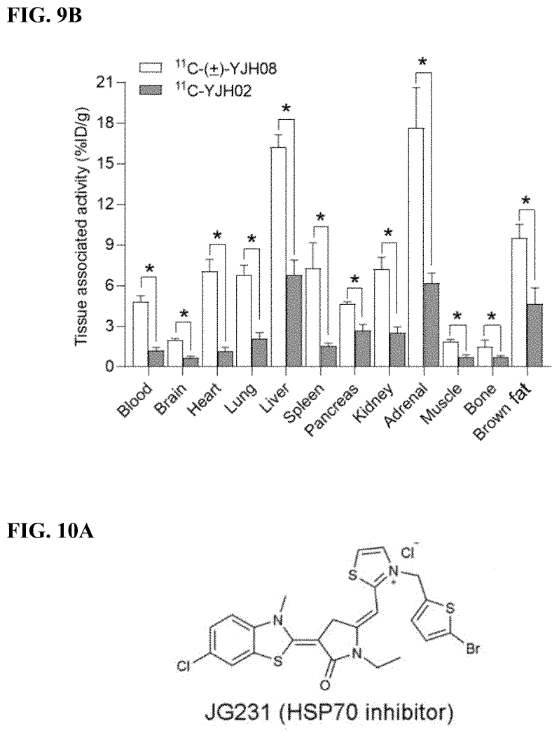

To understand the impact of the C5 moiety on in vivo biodistribution, the phenolic precursor to YJH02 (which bears a carbonyl at the C5 position) was radiolabeled via alkylation with ¹¹C-methyl iodide. The final decay corrected radiochemical yield from 18,500 MBq of ¹¹C-MeI was approximately 4,670±534 MBq, or 70±8% (n=4). The radiochemistry purity was greater than 95%. A biodistribution study was conducted 20 min post injection in mice to determine specific binding in tissues (FIG. 9A and Table 10).

TABLE 10

| | |
|---|---|
| Vehicle | Dex-CD (10 mg/kg) |

| Tissue | Mean ± SD | Mean ± SD | P value |
|---|---|---|---|
| Blood | 1.24 ± 0.1 | 1.07 ± 0.1 | 0.1 |
| Brain | 0.66 ± 0.1 | 0.34 ± 0.07 | 0.001 |
| Heart | 1.15 ± 0.2 | 0.97 ± 0.1 | 0.2 |
| Lung | 2.07 ± 0.4 | 1.71 ± 0.2 | 0.1 |
| Liver | 6.78 ± 1.1 | 3.75 ± 0.8 | 0.0001 |
| Spleen | 1.55 ± 0.1 | 0.76 ± 0.1 | 0.008 |
| Pancreas | 2.70 ± 0.4 | 1.76 ± 0.4 | 0.008 |
| Kidnev | 1.38 ± 0.4 | 1.28 ± 0.1 | 0.06 |
| Stomach | 2.51 ± 0.4 | 1.81 ± 0.3 | 0.02 |
| Sm. Int. | 10.88 ± 2.7 | 5.80 ± 2.5 | 0.01 |
| Lg. Int. | 1.63 ± 0.4 | 1.06 ± 0.3 | 0.97 |
| Adrenal | 6.17 ± 0.7 | 2.37 ± 1.0 | 0.0009 |
| Muscle | 0.74 ± 0.1 | 0.57 ± 0.1 | 0.07 |
| Bone | 0.70 ± 0.1 | 0.76 ± 0.1 | 0.4 |
| Brown Fat | 4.65 ± 1.1 | 3.64 ± 1.0 | 0.18 |

Table 10 summarizes biodistribution data collected 20 minutes after injection of ¹¹C-YJH02 among two treatment arms receiving vehicle or one intraperitoneal injection of the water-soluble dexamethasone-cyclodextrin complex at 10 mg/kg one hour prior to radiotracer administration. The P value indicates the result of an unpaired, two-tailed Student's t test between the respective treatment group and vehicle (n=5/group).

Tracer uptake was suppressed in several tissues from mice treated intraperitoneally with Dex-CD compared to those treated with vehicle. Moreover, the overall pattern of tracer uptake was qualitatively similar to what was observed with (±)-¹¹C-YJH08, with the highest uptake observed in the liver, large and small intestine, kidney, and heart. Directly comparing the uptake values in % ID/g of ¹¹C-YJH02 versus (±)-¹¹C-YJH08 showed that ¹¹C-YJH02 levels were generally lower (FIG. 9B). A stark difference in brain uptake was noted, and ¹¹C-YJH02 uptake was approximately three fold lower than what was observed for (±)-¹¹C-YJH08 (FIG. 9B). These data point to the importance of the benzyl fluoride group for high affinity interactions with GR and access to GR within tissues of high importance to GR (patho)biology.

A Rodent Dosimetry Study shows that Human Equivalent Organ Doses for (f)-¹¹C-YJH08 align with other Carbon-11 and Fluorine-18 Radiotracers currently in Human use. To explore the potential of (±)-¹¹C-YJH08 for human translation, a rodent dosimetry study was conducted in male and female C57B16/J mice (n=4/gender). The human equivalent doses for an average adult male (73 kg) or an average adult female (60 kg) were calculated using OLINDA 2.0 incorporating ICRP103 tissue weighting factors based on region of interest analysis from 90 min dynamic PET acquisitions (Table 11). Table 11 summarizes mouse dosimetry data obtained for (±)-11C-YJH08. The values were calculated from a 90 min dynamic PET acquisition in intact male or female C57B16/J mice. The organs with the highest absorbed doses were the heart, stomach wall, liver, and thymus. All these tissues abundantly express GR, and in the case of the liver, the hepatobiliary mode of clearance (a slower process than renal clearance) for (±)-¹¹C-YJH08 may contribute to the absorbed dose. The estimated whole-body effective dose to an adult male was 0.0067±0.002 mSv/MBq and 0.0065±0.004 mSv/MBq to an adult female. These values are comparable to those of other carbon-11 radiotracers that have been studied in humans, for example ¹¹C-Pittsburgh Compound B, and smaller than a calculated effective dose for ¹⁸F-fluorodeoxyglucose (see, O'Keefe G J, et al. J Nucl Med. 2009; 50:309-15; Quinn B, et al. BMC Med Imaging. 2016; 16:41; Scheinin N M, et al. J Nucl Med. 2007; 48:128-33).

TABLE 11

Absorbed Dose (mGy/MBq) or
Equivalent Dose (mSv/MBq)

| Organ | Adult male (73 kg) | Adult female (60 kg) |
|---|---|---|
| Adrenals | 0.00533 ± 0.000748 | 0.00578 ± 0.000156 |
| Brain | 0.00140 ± 0.000022 | 0.00229 ± 0.000078 |
| Breasts | | 0.00286 ± 0.000129 |
| Esophagus | 0.00831 ± 0.004249 | 0.00544 ± 0.000439 |
| Eyes | 0.00194 ± 0.000092 | 0.00223 ± 0.000061 |
| Gallbladder Wall | 0.00602 ± 0.000876 | 0.00459 ± 0.000041 |
| Left colon | 0.00374 ± 0.000769 | 0.00437 ± 0.000314 |
| Small Intestine | 0.00325 ± 0.000446 | 0.00325 ± 0.000114 |
| Stomach Wall | 0.01628 ± 0.005962 | 0.01925 ± 0.002944 |
| Right colon | 0.00367 ± 0.000548 | 0.00339 ± 0.000070 |
| Rectum | 0.00249 ± 0.000093 | 0.00280 ± 0.000087 |
| Heart Wall | 0.06753 ± 0.050725 | 0.02570 ± 0.005677 |
| Kidneys | 0.00714 ± 0.000174 | 0.00785 ± 0.001344 |
| Liver | 0.01568 ± 0.001377 | 0.01863 ± 0.001258 |
| Lungs | 0.01350 ± 0.011132 | 0.00909 ± 0.000339 |
| Pancreas | 0.00665 ± 0.002374 | 0.00545 ± 0.000182 |
| Ovaries | | 0.00288 ± 0.000090 |
| Prostate | 0.00250 ± 0.000105 | |
| Salivary Glands | 0.00226 ± 0.000156 | 0.00241 ± 0.000068 |
| Red Marrow | 0.00310 ± 0.000771 | 0.00289 ± 0.000101 |
| Osteogenic Cells | 0.00223 ± 0.000391 | 0.00217 ± 0.000069 |
| Spleen | 0.00378 ± 0.000828 | 0.00432 ± 0.000300 |
| Testes | 0.00204 ± 0.000046 | |
| Thymus | 0.01004 ± 0.005999 | 0.00498 ± 0.000454 |
| Thyroid | 0.00355 ± 0.001070 | 0.00297 ± 0.000118 |
| Urinary | 0.00247 ± 0.000136 | 0.00288 ± 0.000114 |
| Bladder Wall | | |
| Uterus | | 0.00284 ± 0.000085 |
| Total Body | 0.00355 ± 0.000856 | 0.00366 ± 0.000121 |
| Effective Dose | 0.00673 ± 0.002757 | 0.00649 ± 0.000433 |
| (mSv/MBq) | | |

The data reported here support the clinical translation of (±)-¹¹C-YJH08 to study GR biology in humans. The radiosynthesis of (±)-¹¹C-YJH08 was higher yielding than for (±)—¹⁸F-YJH08, and biodistribution studies showed equivalence to (±)—¹⁸F-YJH08. Importantly, the average final production yield of 2590±103.6 MBq is sufficient for subsequent clinical translation. Somewhat unexpectedly, both the (R) and (S) enantiomers of YJH-08 were predicted to have equal affinities for GR, and their in vivo patterns of biodistribution were mutually indistinguishable. Corticosteroid withholding and supplementation studies revealed evidence of specific binding for (±)-¹¹C-YJH08 in vivo. Radioligand suppression was not achieved to equivalent extents in all tissues, although this could reflect an inability to safely saturate GR in vivo. Notably, dramatic suppression of (±)-¹⁸F-YJH08 uptake was observed in the adipose tissue of an adipocyte specific GR knockout mouse, which adds support to a model for GR specific binding in vivo for this ligand structure.

Analysis of the YJH08 structure activity relationship in vitro established the importance of the benzyl moiety on the C5 position to GR affinity. Moreover, a biodistribution study with ¹¹C-YJH02 showed the importance of the benzyl fluoride group to tissue uptake in vivo, especially in compartments like the brain that can be challenging to access. The altered biodistribution of ¹¹C-YJH02 may be attributable to somewhat lower affinity for GR (Table 9). In addition, lower blood pool activity was observed for ¹¹C-YJH02 at 20 min post injection, which could indicate that lower tissue uptake is due to altered pharmacokinetics. Finally, a dosimetry study estimated the human equivalent doses of (±)-¹¹C-YJH08 to be within the range of carbon-11 and fluorine-18 radiotracers that are in human use. Collectively, these data support the feasibility of human imaging studies with (±)-¹¹C-YJH08 to study GR biology. Moreover, with experimental GR modulators entering clinical trials (e.g., ORIC-101), (±)-¹¹C-YJH08 could be a useful companion diagnostic to identify tissues with altered GR expression and assess drug pharmacodynamics as has been previously done with radioligands targeting the androgen receptor and the estrogen receptor (see, Rathkopf D E, et al. J Clin Oncol. 2013; 31:3525-30; Kurland B F, et al. C/in Cancer Res. 2017; 23:407-415; Rew &, et al. J Med Chem. 2018; 61:7767-7784).

### Example 8. ¹⁸F-YJH08 Reveals Tissue Specific Modulation of GR Expression by JG231, a Potent HSP70 Inhibitor

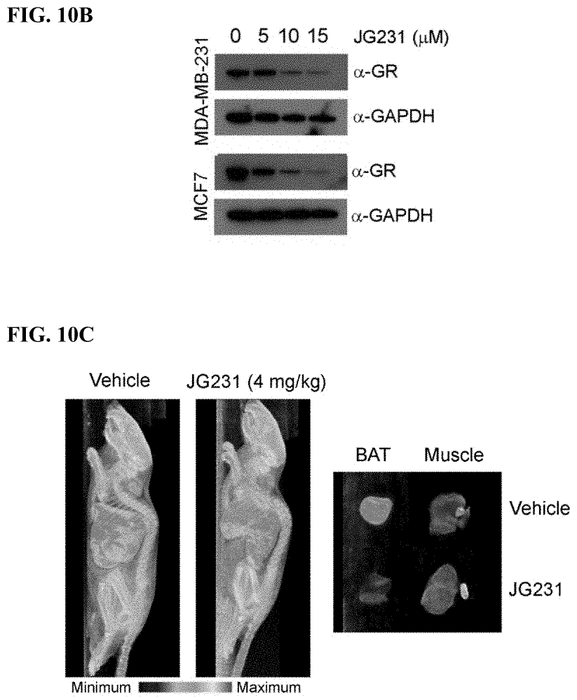

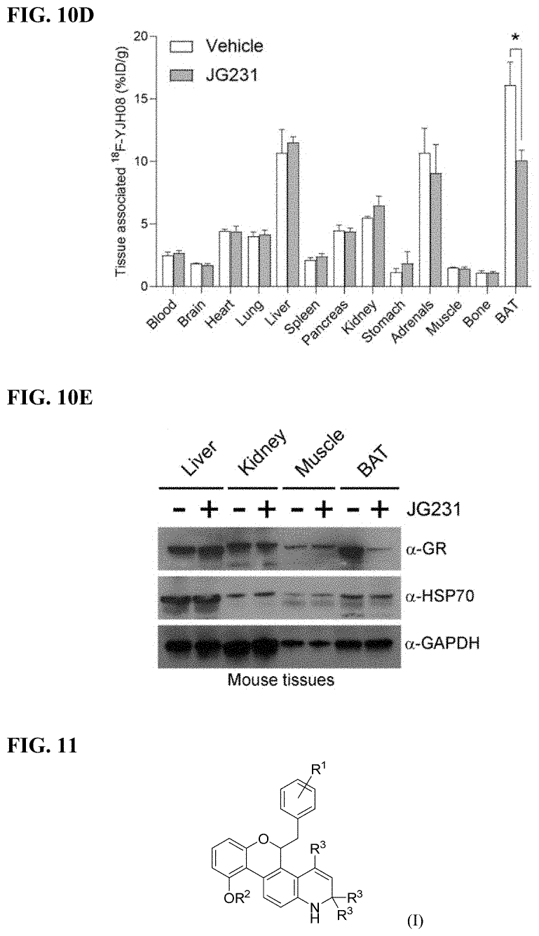

The limitations of broad-spectrum GR modulators like corticosteroids are well known, and the severe side effects associated with their long term use (e.g. diabetes mellitus, osteoporosis) motivated drug development campaigns starting in the 1990s to identify safer GR modulators that selectively modulate GR activity (Sundahl, et al. Pharmacol. Ther. 2015, 152, 28-41). The study in this example was initiated with the hypothesis that ¹⁸F-YJH08 PET could complement these ongoing efforts by revealing tissue specific drug-GR interactions. To test this concept, the biodistribution of ¹⁸F-YJH08 was evaluated in mice after treatment with JG231 (FIGS. 10A and 10B). JG231 is a potent HSP70 inhibitor that has been shown to induce GR degradation (Shao, et al. J. Med. Chem. 2018, 61 (14), 6163-6177). JG231 has a well-defined pharmacokinetic profile, and prior experience with this molecule showed it to have antitumor activity in prostate cancer mouse models (Moses, et al. Cancer Res. 2018, 78 (14), 4022-4035). Using the same treatment strategy, mice were subjected to 4 mg/kg/day for three days intraperitoneally, and subsequently treated with ¹⁸F-YJH08. On PET, the only visually obvious alterations in radiotracer distribution were noted in the supraspinal brown fat depot and in a portion of the abdomen that was interpreted to be the greater omentum (FIG. 10C). In these regions, it appeared that JG231 suppressed radiotracer binding, perhaps indicative of GR degradation. Post mortem imaging of the supraspinal brown fat depot more clearly showed that JG231 dramatically reduced ratiotracer binding compared to the same tissue harvested from mice treated with vehicle (FIG. 10C). Biodistribution studies also suggested that JG231 most significantly induced GR degradation in brown fat (FIG. 10D). Immunoblot studies corroborated the biodistribution data, showing approximately ˜50% reduction in GR expression levels due to JG231 treatment in brown fat, and no change in GR levels from other tissues, for example, kidneys, liver, and muscle (FIG. 10E). This is believed to be the first example of tissue specific GR degradation. More generally, these data show that ¹⁸F-YJH08 PET could be applied to identify therapies with unexpected tissue tropism or prospectively engineer desirable tissue specificity into experimental GR modulators.

### VI. EXEMPLARY EMBODIMENTS

Exemplary embodiments provided in accordance with the presently disclosed subject matter include, but are not limited to, the claims and the following embodiments:
1. A compound according to Formula I:

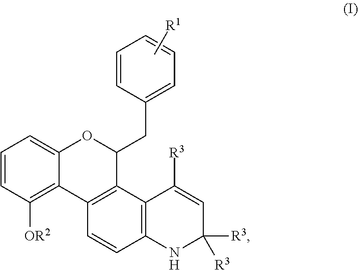

or a pharmaceutically acceptable salt thereof,
wherein:
R¹ is selected from the group consisting of halogen, C₁₋₆ alkyl, and C₆₋₁₂ aryl, each of which is optionally radiolabeled;
R² is C₁₋₆ alkyl, which is optionally radiolabeled; and
each R³ is independently C₁₋₆ alkyl.
2. The compound of embodiment 1, or a pharmaceutically acceptable salt thereof, wherein at least one of R¹ and R² is radiolabeled.
3. The compound of embodiment 1 or embodiment 2, or a pharmaceutically acceptable salt thereof, wherein R¹ is radiolabeled halogen.
4. The compound of embodiment 3, or a pharmaceutically acceptable salt thereof, wherein R¹ is —¹⁸F.
5. The compound of embodiment 1 or embodiment 2, or a pharmaceutically acceptable salt thereof, wherein R² is —¹¹CH₃.
6. The compound of any one of embodiments 1-5, or a pharmaceutically acceptable salt thereof, wherein each R³ is methyl.
7. The compound of any one of embodiments 1-6, or a pharmaceutically acceptable salt thereof, having a structure according to Formula Ia:

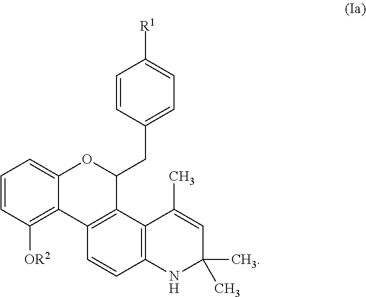

8. The compound of embodiment 1 or embodiment 7, or a pharmaceutically acceptable salt thereof, wherein R¹ is —¹⁸F and R² is —CH₃.
9. The compound of embodiment 1 or embodiment 7, or a pharmaceutically acceptable salt thereof, wherein R¹ is —F and R² is —¹¹CH₃.
10. The compound of embodiment 1 or embodiment 7, which is selected from the group consisting of:

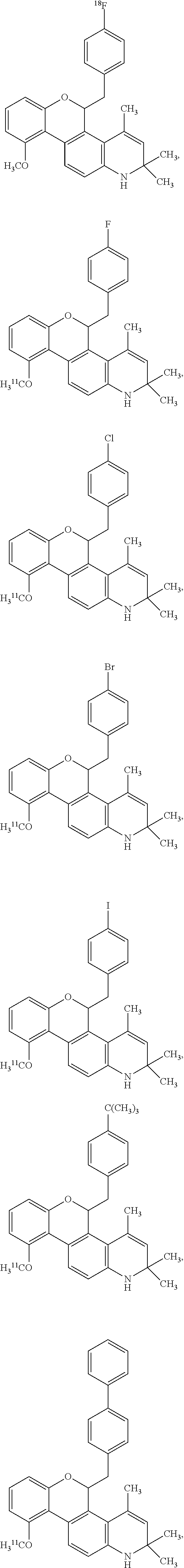

and pharmaceutically acceptable salts thereof
11. A composition comprising a compound according to any one of embodiments 1-10 and a pharmaceutically acceptable excipient.
12. A method for detecting the expression of glucocorticoid receptor (GR) in a subject, the method comprising:
(i) administering to the subject an effective amount of a compound according to any one of embodiments 1-10, or a pharmaceutically acceptable salt thereof,
wherein at least one of R¹ and R² in the compound is radiolabeled, and wherein the compound is administered to the subject under conditions sufficient for binding of the compound to GR in the subject; and
(ii) detecting the compound in the subject by positron emission tomography, thereby detecting the expression of GR in the subject.
13. The method of embodiment 12, wherein GR expression is detected in brain tissue, adipose tissue, kidney tissue, or a combination thereof in the subject.
14. The method of embodiment 13, wherein GR expression is detected in brain tissue.
15. The method of embodiment 12, wherein GR expression is detected in cancerous tissue in the subject.
16. The method of embodiment 15, wherein GR expression is detected in prostate cancer in the subject.
17. The method of embodiment 15 or embodiment 16, further comprising administering to the subject a therapeutically effective amount of a GR modulator, thereby treating cancer in the subject.
18. The method of any one of embodiments 12-17, wherein the compound is

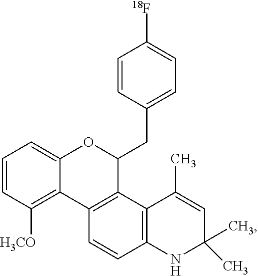 [SMILES: COc1cccc2c1-c1ccc3c(c1C(Cc1ccc([18F])cc1)O2)C(C)=CC(C)(C)N3]

or a pharmaceutically acceptable salt thereof.
19. The method of any one of embodiments 12-18, wherein the compound in step (i) is administered to the subject in an amount ranging from about 0.1 mg/kg to about 200 mg/kg.
20. A method for preparing a compound according to Formula II:

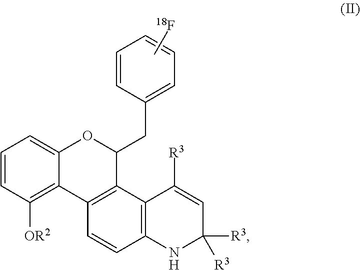

or a pharmaceutically acceptable salt thereof, the method comprising:
(a) forming a reaction mixture comprising an ¹⁸F-fluoride salt, a copper salt, and compound according to Formula IV:

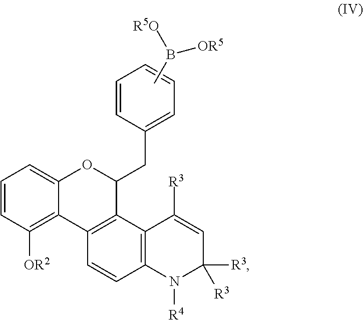

(b) maintaining the reaction mixture under conditions sufficient to form a compound according to Formula III:

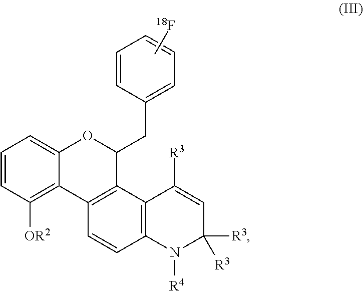

and
(c) converting the compound of Formula III to the compound of Formula II;
wherein:
R² is C₁₋₆ alkyl;
each R³ is independently C₁₋₆ alkyl;
R⁴ is an amine protecting group; and
each R⁵ is independently selected from the group consisting of hydrogen and C₁₋₆ alkyl, or
both R⁵ and the grouping —O—B—O— are taken together to form an optionally-substituted 5- or 6-membered ring.
21. The method of embodiment 20, wherein the copper salt is Cu(py)₄(OTf)₂.
22. The method of embodiment 20 or embodiment 21, wherein the ¹⁸F-fluoride salt is potassium ¹⁸F-fluoride.
23. The method of any one of embodiment 20-22, wherein both R⁵ and the grouping —O—B—O— are taken together to form a boronic acid pinacol ester.
24. The method of any one of embodiments 20-24, wherein R² and R³ are methyl.

Although the foregoing has been described in some detail by way of illustration and example for purposes of clarity and understanding, one of skill in the art will appreciate that certain changes and modifications can be practiced within the scope of the appended claims. In addition, each reference provided herein is incorporated by reference in its entirety to the same extent as if each reference was individually incorporated by reference.

What is claimed is:

1. A compound according to Formula I: 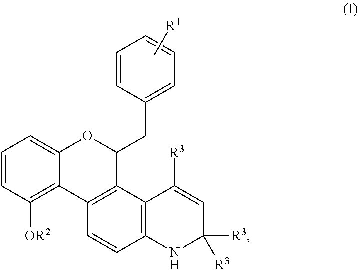
or a pharmaceutically acceptable salt thereof,
wherein:
R¹ is selected from the group consisting of radiolabeled halogen, unlabeled halogen, radiolabeled C₁₋₆ alkyl, unlabeled C₁₋₆ alkyl, radiolabeled C₆₋₁₂ aryl, and unlabeled C₆₋₁₂ aryl;
R² is selected from the group consisting of unlabeled C₁₋₆ alkyl and radiolabeled C₁₋₆ alkyl; and
each R³ is independently C₁₋₆ alkyl.

2. The compound of claim 1, or a pharmaceutically acceptable salt thereof, wherein at least one of R¹ and R² is radiolabeled.

3. The compound of claim 1, or a pharmaceutically acceptable salt thereof, wherein R¹ is radiolabeled halogen.

4. The compound of claim 3, or a pharmaceutically acceptable salt thereof, wherein R¹ is —¹⁸F.

5. The compound of claim 1, or a pharmaceutically acceptable salt thereof, wherein R² is —¹¹CH₃.

6. The compound of claim 1, or a pharmaceutically acceptable salt thereof, wherein each R³ is methyl.

7. The compound of claim 1, or a pharmaceutically acceptable salt thereof, having a structure according to Formula Ia:

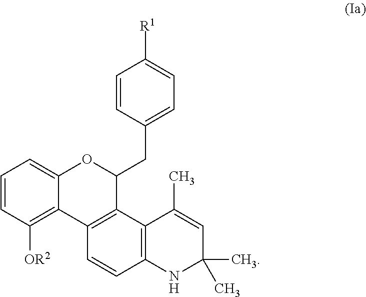

8. The compound of claim 1, or a pharmaceutically acceptable salt thereof, wherein R¹ is —¹⁸F and R² is —CH₃.

9. The compound of claim 1, or a pharmaceutically acceptable salt thereof, wherein R¹ is —F and R² is —¹¹CH₃.

10. The compound of claim 1, which is selected from the group consisting of: 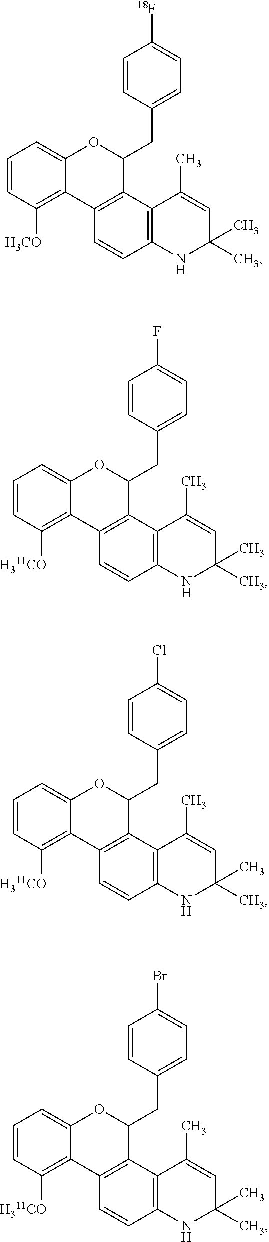 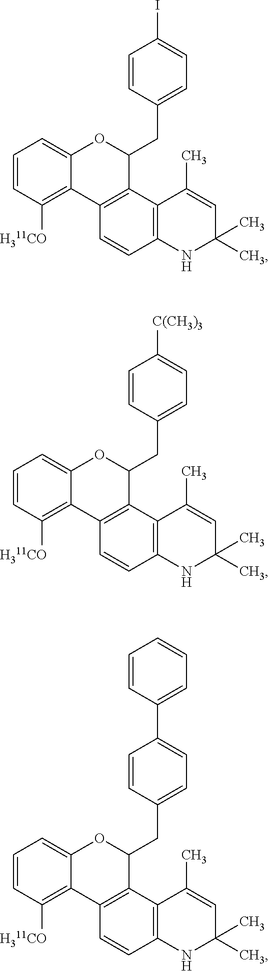
and pharmaceutically acceptable salts thereof.

11. A composition comprising a compound according to claim 1 and a pharmaceutically acceptable excipient.

12. A method for detecting the expression of glucocorticoid receptor (GR) in a subject, the method comprising:
(i) administering to the subject an effective amount of a compound according to claim 1, or a pharmaceutically acceptable salt thereof,
wherein at least one of R¹ and R² in the compound is radiolabeled, and wherein the compound is administered to the subject under conditions sufficient for binding of the compound to GR in the subject; and
(ii) detecting the compound in the subject by positron emission tomography, thereby detecting the expression of GR in the subject.

13. The method of claim 12, wherein GR expression is detected in brain tissue, adipose tissue, kidney tissue, or a combination thereof in the subject.

14. The method of claim 13, wherein GR expression is detected in brain tissue.

15. The method of claim 12, wherein GR expression is detected in cancerous tissue in the subject.

16. The method of claim 15, wherein GR expression is detected in prostate cancer in the subject.

17. The method of claim 15, further comprising administering to the subject a therapeutically effective amount of a GR modulator, thereby treating cancer in the subject.

18. The method of claim 12, wherein the compound is 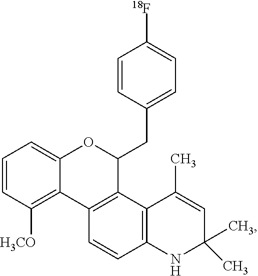 [SMILES: COc1cccc2c1-c1ccc3c(c1C(Cc1ccc([18F])cc1)O2)C(C)=CC(C)(C)N3]
or a pharmaceutically acceptable salt thereof.

19. The method of claim 12, wherein the compound in step (i) is administered to the subject in an amount ranging from about 0.1 mg/kg to about 200 mg/kg.

20. A method for preparing a compound according to Formula II: 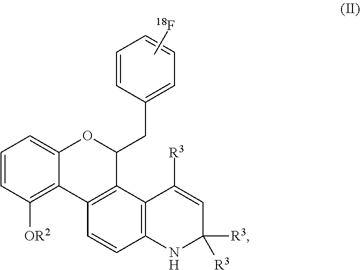
or a pharmaceutically acceptable salt thereof, the method comprising:
(a) forming a reaction mixture comprising an ¹⁸F-fluoride salt, a copper salt, and compound according to Formula IV: 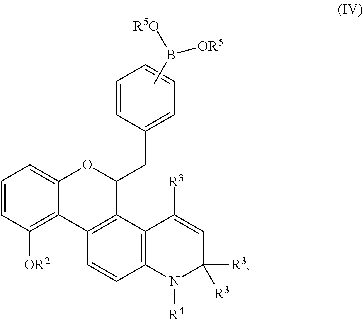
(b) maintaining the reaction mixture under conditions sufficient to form a compound according to Formula III: 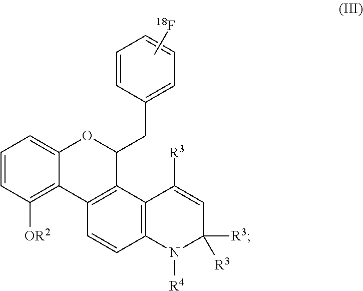
and
(c) converting the compound of Formula III to the compound of Formula II;
wherein:
R² is C₁₋₆ alkyl;
each R³ is independently C₁₋₆ alkyl;
R⁴ is an amine protecting group; and
each R⁵ is independently selected from the group consisting of hydrogen and C₁₋₆ alkyl, or
both R⁵ and the grouping —O—B—O— are taken together to form an optionally-substituted 5- or 6-membered ring.
